(19) United States
(12) Patent Application Publication
(10) Pub. No.: US 2002/0065213 A1
(43) Pub. Date: May 30, 2002

(54) METHODS AND COMPOSITIONS FOR NONVIRAL GENE DELIVERY

(76) Inventor: ROBERT J. DEBS, MILL VALLEY, CA (US)

Correspondence Address: Tracy J. Dunn I.P., 2000 Crystal Springs Road, Suite 2243, San Bruno, CA, 94066, US

(21) Appl. No.: 09/232,175

(22) Filed: Jan. 15, 1999

Related U.S. Application Data
(60) Provisional application No. 60/071,598, filed on Jan. 16, 1998.

(51) Int. Cl. A61K 48/00; A61K 38/17; C12P 21/02; C12N 5/06; C12N 5/08; C12N 15/87

(52) U.S. Cl. 514/2; 514/8; 514/12; 514/44; 530/383; 435/69.1; 435/455; 435/352; 435/366

(57) ABSTRACT

Methods and compositions are described for the efficient delivery and expression of recombinant polynucleotides in animal cells in vitro or in vivo. In particular, a comprehensive approach of non-viral gene delivery is provided that has been optimized with regards to the vectors delivered, maximizing the amount and duration of gene expression, and methods of conditioning the patient to enhance the efficiency of gene delivery. The described methods find application in both gene therapy, and the functional analysis of cloned gene products in vivo.

This application is a continuation-in-part of U.S. Provisional Application Ser. No. 60/071,598, filed Jan. 16, 1998.

### INTRODUCTION

The present invention is relevant to the fields of biochemistry, cell biology, and molecular genetics. In particular, novel compositions and methods are reported which efficiently deliver polynucleotides or other bioactive materials to mammalian cells in vitro and in vivo, increase the levels of expression in vitro and in vivo, increase tissue specific expression, and prolong the duration of expression in vitro and in vivo. Additionally, the present invention relates methods for the optimization of efficiency of gene delivery.

### BACKGROUND

Molecular biology techniques allow for the efficient and precise engineering of polynucleotides. However, techniques for efficiently delivering, and subsequently expressing, engineered polynucleotides that contain genes or cDNAs, as well as the host factors which control gene delivery and expression in vivo, remain less mature. For example, a wide variety of viral vectors have been developed to deliver polynucleotides to mammalian cells. These vectors are relatively efficient tools for gene delivery in vitro but are often constrained by physical and biological considerations particularly in the in vivo setting. In particular, packaging considerations often limit the amount of polynucleotide that can be delivered by viral vectors, and virus biology often limits the variety of cells that are suitable for virally mediated gene delivery and chromosomal repair or manipulation (see generally, Friedman, June 1997, Scientific American, pp. 97-101).

Additionally, viruses (e.g., Epstein Barr Virus) have been linked to tumor formation in man, and virally encoded proteins such as large T antigen, EBNA-1, etc., are known to transform cells in culture or cause tumors in vivo (Wilson et al., 1996, EMBO, 15:3117-3126; Cooper et al., 1997, Proc. Natl. Acad. Sci., USA, 94:6450-6455).

Another factor that may hinder the broader application of viral mediated gene delivery in vivo, is the fact that the host immune system can significantly impact the efficiency of viral gene delivery, particularly following readministration of viral vectors into immuno-competent hosts.

An alternative method of delivering genetically engineered polynucleotides to cells involves the use of liposomes (see generally, Felgner, June 1997, Scientific American, pp. 102-106). The phospholipid bilayer of the liposome is typically made of materials similar to the components of the cell membrane. Thus, polynucleotides associated with liposomes (either externally or internally) can be delivered to the cell when the liposomal envelope fuses with the cell membrane. More typically, the liposome/polynucleotide complex will be endocytosed into the cell. After endocytosis the internal contents of the endosome, including the delivered polynucleotide, may be released into the cytoplasm.

Classical liposome-mediated polynucleotide delivery is limited by the relatively small internal volume of the liposome. Thus, it is difficult to effectively entrap sufficient quantities of polynucleotide within liposomal formulations.

Researchers have tried to compensate for the inherent inefficiency of liposomal encapsulation by adding positively charged amphipathic lipid moieties to liposomal formulations. In principle, the positively charged groups of the amphipathic lipids ion-pair with the negatively charged polynucleotides and increase the amount of association between the polynucleotides and the lipidic particles. The enhanced association presumably promotes binding of the nucleic acid to the lipid bilayer. Several cationic lipid products are commercially available that are useful for the introduction of nucleic acid into mammalian cells. In particular, LIPOFECTIN™ (DOTMA, which consists of a monocationic choline head group which is attached to diacylglycerol (see generally, U.S. Pat. No. 5,208,036 to Epstein et al.); TRANSFECTAM™ (DOGS) a synthetic cationic lipid with lipospermine head groups (Promega, Madison, Wis.); DMRIE and DMRIE•HP (Vical, La Jolla, Calif.); DOTAP™ (Boehringer Mannheim (Indianapolis, Ind.), DOTIM, and Lipofectamine (DOSPA) (Life Technology, Inc., Gaithersburg, Md.) have been widely used. In addition, cationic polymers, including for example polyethylenimine (22 kDa PEI, ExGene 500) are also available and have been shown useful for introduction of nucleic acid into mammalian cells (see Goula et al., 1998, Gene Ther. 5:712-717; and WO 95/FR/00914).

Properly employed, the above compounds mediate the delivery of nucleic acids into cells cultured in vitro. However, significant levels of cellular toxicity have been associated with commercially available cationic lipids. Consequently, most commercially available cationic lipids are not well suited for in vivo gene delivery applications at their present level of gene transfer efficiency. Additionally, conventional techniques for cationic lipid-mediated gene delivery generally provide lower levels of in vivo gene expression relative to those typically obtained using certain viral vectors, such as adenovirus, and expression is only transient and relatively short lived.

### SUMMARY OF THE INVENTION

The present invention relates to methods and compositions for nonviral gene delivery to animal cells in vitro and in vivo. In particular, methods and compositions are described that significantly increase the expression levels of nonvirally delivered genes, and allow for sustained expression in vivo.

Accordingly, one aspect of the present invention is a pair of novel expression vectors. The first vector incorporates a gene encoding a cellular retention activity, e.g., EBNA-1, in a vector lacking either EBV family of repeat (FR) DNA sequences, or an intact oriP (FR plus the region of dyad symmetry). Preferably, the vectors of the present invention will incorporate a strong viral or mammalian promoter element, such as, for example, the human cytomegalovirus (HCMV) IE-1 promoter enhancer element that controls the expression of the gene of interest. In this instance, the EBNA-1 gene is expressed by the HCMV IE-1 promoter/enhancer region.

A second vector contains the gene to be delivered, i. e., the gene of interest, and additionally encodes a control element that directly or indirectly interacts with the retention activity encoded by the first vector. For example, where the first vector encodes an EBNA-1 gene, the second vector will typically encode an EBV-FR DNA sequence, or, optionally, can encode an intact oriP that allows replication in human cells. Alternatively, these sequences can be appended to the first vector, or vice-versa, to generate a single vector encoding a cellular or nuclear retention activity, a control element that is recognized by the retention activity, and a expression cassette containing the gene of interest.

Optionally, the second vector can incorporate a modified EBV-FR region that lacks an intact or functional oriP sequence. Typically, such a vector will lack the region of dyad symmetry (DS); however, where vector replication in primate or human cells is desired, such vectors will generally contain an intact oriP sequence, or functional equivalent thereof.

When delivered together, the above vectors markedly increase the levels and duration of expression in the target cell. Accordingly, an additional embodiment of the present invention is the codelivery of the above vectors to target cells. The vectors can be delivered as “naked” DNA, or in conjunction with chemicals or cofactors that protect the DNA or facilitate gene delivery into the target cells. As such, an additional aspect of the present invention is a lipid complex comprising a recombinant gene of interest, a recombinant nucleic acid sequence encoding a cellular retention activity, and a recombinant nucleic acid sequence encoding a cellular retention sequence. For the purposes of the present disclosure, a “recombinant” polynucleotide or nucleic acid sequence refers to a naturally occurring or genetically manipulated sequence that is present on an engineered vector (i.e., a vector containing a non-naturally occurring organization of sequence elements).

Another embodiment of the present invention is the use of the above-described sequences in a lipid or polymer complex to deliver a gene of interest to eukaryotic, preferably mammalian, organisms or cells either in vitro or in vivo.

An additional embodiment of the present invention is a method of increasing the levels of expression of a delivered gene in target cells by substantially simultaneously delivering (i.e., simultaneously or within about 24 to 48 hours), or previously delivering, a gene encoding a cellular retention activity to the target cells.

Another embodiment of the present invention is a method of increasing the duration of expression of a delivered gene in target cells by delivering a gene encoding a cellular retention activity and nuclear binding sequence to the target cells before, or preferably substantially simultaneously with, or several hours prior to, the introduction of the delivered gene.

Additional embodiments of the present invention include gene delivery compositions, and methods of making the same, that incorporate diluents, solutions, and compounds that are suitable for use in vivo. Examples of such diluents, solutions, and compounds include, but are not limited to, lactated Ringers, sterile I.V. grade dextrose solutions, cationic polymers, lipid emulsions, dextrans (such as dextran 40), protamine sulfate, albumin, human serum, pharmaceutically useful solid supports such as collagen beads and supports, microcarrier beads, and polymeric and time release formulations and/or suspensions thereof, and the like as well as any and all combinations or mixtures of the

Additional embodiments of the present invention include methods of treating the gene delivery recipient with one or more suitable compounds prior to, during, and/or subsequent to gene delivery. Particular examples of such compounds include dexamethasone, corticosteroids, ammonium chloride, chloroquine, quinine, quinidine, 4-APP (4-aminopyrazolo[3,4d]pyrimidine), retinoic acid, valproiac acid, mixtures of the above, e.g., dexamethasone together with valproiac acid, and the like.

### DESCRIPTION OF THE FIGURES

FIG. 1 shows the levels of luciferase expression in the hearts and lungs of test animals at 24 hours or 7 days after the introduction of the disclosed vectors in vivo.

FIG. 2 shows the levels of luciferase expression in the hearts and lungs of test animals at 24 hours, 6 weeks, or 14 weeks after the introduction of the disclosed vectors in vivo.

FIGS. 3a and 3b show that luciferase expression in the hearts and lungs of test animals continues and is significantly enhanced after redosaging at 31 days after the introduction of the disclosed vectors in vivo (3a). FIG. 3b shows that cationic lipid DNA complex (hereinafter “CLDC”) delivery of luc-FR-EBNA-1 produced significant luciferase expression after two prior injections of CAT-EBNA-1 (each injection spaced three weeks apart).

FIG. 4 shows that formulating CLDC in the presence of different diluents can affect the levels of CLDC-mediated luciferase expression in vivo.

FIG. 5 shows that CLDC mediated luciferase expression in vivo differs among different strains of mice.

FIG. 6 shows that pretreating animals with various agents can affect the levels of CLDC mediated luciferase expression in vivo.

FIG. 7 shows that CLDC mediated delivery of the GM-CSF, angiostatin, and p53 genes resulted in significant anti-metastatic effects in vivo.

FIG. 8 shows how CLDC mediated delivery was used to identify a novel anti-tumor gene function for CC3.

FIG. 9 is a schematic diagram of expression vectors p4329, p4379, p4395, p4402, and p4458.

### DETAILED DESCRIPTION OF THE INVENTION

The internalization and expression of genes delivered by non-viral mediated methods involves a variety of biological processes. Each of these processes provides an opportunity for optimizing both the level of expression of exogenously introduced polynucleotides, and the duration of said expression. For example, as charged molecules, biologically relevant polynucleotides do not readily cross the cell membrane. Accordingly, any mechanism that enhances a polynucleotide's ability to enter a target cell will typically enhance the efficiency of gene delivery. However, after a polynucleotide enters the cell, expression of the encoded gene is far from assured. After internalization, the polynucleotide must typically free itself from any cellular factors (e.g., the endosome/lysosome) that were involved in the internalization process, and then find its way into the nucleus where the requisite transcriptional and splicing machinery are typically situated. Once in the nucleus, the polynucleotide must generally directly or indirectly associate with the chromosome, or other nuclear factors such as the nuclear matrix of the nuclear envelope, in order remain in the nucleus and continue expressing the desired product. Yet another challenge in gene delivery is obtaining tissue specific expression of a polynucleotide.

a. Vectors For Gene Delivery

The present invention describes a novel approach to non-viral gene delivery that comprehensively incorporates technology that addresses the above considerations. For example, the presently described vectors are generally episomal vectors and will preferably encode, in addition to at least one copy of the gene of interest, at least one promoter/enhancer region for expressing the gene of interest; optionally, an origin of replication functional in eucaryotic cells or an FR-like sequence; operably positioned splice donor and splice acceptor regions; and at least one or more nuclear retention sequences and/or one or more cellular retention sequences. Optionally, the vector also encodes a cellular retention activity (CRA) and/or nuclear retention activity (NRA). However, in an alternative, preferred embodiment, the CRA and/or NRA is encoded on a separate vector.

i. CRA/CRS Systems

Given that many viruses must also overcome the above outlined obstacles to productively infect a host cell, viral biology has been exploited as one aspect of the invention to construct appropriate synthetic proxies.

For example, Epstein-Barr virus (EBV), like other episomally replicating viruses, maintains its genome as a replicating episomal plasmid in infected cells. Most of the EBV genome is present as a supercoiled DNA of approximately 172,000 bp. EBV provides the EBV nuclear antigen 1 (EBNA-1) to facilitate the replication of its plasmid. EBNA-1 is a viral DNA binding protein that binds in a site-specific fashion to EBV DNA sequences which together constitute the viral DNA origin of replication (oriP). EBV oriP contains two non-continuous regions. One region consists of approximately 20 tandem imperfect copies of a 30 bp sequence, the family of repeats (FR), and the other, separated by approximately 1000 bp from the FR, is at most 114 bp and contains a 65 bp region of dyad symmetry (DS). EBV oriP acts in cis to allow the replication and maintenance of recombinant plasmids in cells harboring either the EBV genome or in cells expressing the EBNA-1 coding sequence. Both the region of DS and the FR sequences must be present in cis for replication. In addition, the FR acts as a transcriptional enhancer and is involved in plasmid maintenance both intracellularly and in the nucleus.

In the presence of EBNA-1, plasmids that contain FR but lack the DS are retained only transiently, for a period of 2 to 3 weeks, in cultured cells (D. Reisman et al., 1985, Mol. Cell Biol., 5:1822-1832; D. Reisman et al., 1986, Mol. Cell Biol., 6:3838-3846; Krysan et al., 1989, Mol Cell Biol., 9:1026-1033). Furthermore, in the presence of EBNA-1, very few FR-containing plasmids are retained in cells over the next several days following plasmid delivery (Middleton and Sugden, 1994, J. Virol., 68(6):4067-4071). In addition, while plasmids containing an intact oriP are able to replicate in primate and human cells, they cannot replicate in rodent cells in the presence of EBNA-1 (Yates et al., 1985, Nature, 313:812-814; Krysan and Calos, 1993, Gene, 136:137-143). However, the substitution of large (21 kb) fragments of human genomic DNA for the EBV DS can produce stable maintenance of plasmids containing the FR and coding for EBNA-1 in rodent cells when cultured in vitro (Krysan and Calos, 1993, supra.).

The presently described vectors incorporate a nuclear retention sequence and/or a cellular retention sequence which can also be one in the same. Nuclear retention sequences are a subset of the cellular retention sequences. As used herein the term cellular retention sequence refers to a region that is directly or indirectly recognized and bound by a cellular retention activity or nuclear retention activity which helps the vector remain in target cells. Additionally, the vector can include one or more nuclear retention sequences (such as, for example, DNA sequences that specifically bind to the nuclear matrix, envelope, or cellular chromosomes) that can interact with appropriate cellular features, cellularly encoded factors, or exogenously added or encoded factors to further increase retention of the vector in the nucleus. For the purposes of the present invention, the terms cellular retention activity (CRA) and nuclear retention activity (NRA) refer to a protein, peptide, or DNA sequences, that directly or indirectly interacts with a nuclear retention sequence or cellular retention sequence such that the polynucleotide containing or encoding the nuclear retention sequence or cellular retention sequence displays enhanced levels of expression or enhanced duration of expression relative to a control polynucleotide that does not encode the nuclear and cellular retention sequences.

When used in conjunction with suitable cellular and nuclear retention activities, vectors bearing a nuclear and/or cellular retention sequence exhibit an enhanced duration of expression of the gene of interest. Typically, an enhanced duration of expression is characterized by the fact that the described vectors episomally express detectable levels of the gene of interest in the target cell long-term; e.g., for at least about 20 percent longer than vectors lacking the nuclear retention sequence, more typically at least about 50 percent longer, and preferably at least about 100 percent longer than episomal vectors lacking the nuclear retention sequence. Examples of representative nuclear retention sequences suitable for use in the present invention include, but are not limited to, EBV sequences which bind to the matrix attachment region (MAR), or an acidic domain of the carboxy terminus of HCMV IE-1 (Hill et al., 1988, Cell, 55(3):459-466). Examples of representative cellular retention sequences include, but are not limited to, the EBV-FR sequence (Middleton and Sugden, J. Virol., 1994, 68(6):4067-4071 (p. 4068, ¶5 specifically) and similar sequences. The observed in vivo advantages of incorporating the FR sequence into the described vectors were particularly surprising given that in vitro studies had indicated that FR is a transcriptional enhancer, but similar enhancer activity could not be detected in vivo.

Similarly, vectors having either a cellular or nuclear retention sequence will typically express the gene of interest at about 20 percent higher (and/or longer), more typically at least about 50 percent higher (and/or longer, preferably at least about 100 percent higher (and/or longer), and specifically at least about 3 to 5 fold higher (and/or longer) than otherwise identical vectors lacking either a cellular or nuclear retention sequence, when used in conjunction with the appropriate CRA and/or NRA.

Preferably, the cellular and the nuclear retention sequences (CRS or NRS, respectively) are located within the vector at regions that do not interfere with gene expression. For example, these sequences are preferably not located between the enhancer/promoter region for the gene of interest, and the 5′ end of the gene of interest (i.e., not between the enhancer/promoter region and the region immediately upstream from the AUG start codon of the gene of interest). Although the protein EBNA-1 has been provided as a specific example of a preferred cellular retention activity (CRA), the present invention is in no way limited to this specific protein. In fact, a variety of CRA/CRS and NRA/NRS pairings are deemed suitable for use in the present invention including, but are not limited to, karyopherin (N. Fisher et al., 1997, J. Biol. Chem., 272(7):3999-4005) and other importins/karyopherins, and functional fragments and fusions thereof, and the pairing of a sequence, plasmid, or vector encoding the adenovirus preterminal protein with a second sequence, plasmid, or vector encoding the adenovirus ITR sequences.

The large size of the present vectors allows a single vector to encode both the CRS or NRS, and a gene encoding the CRA and/or NRA. However, it can be preferable to only transiently express the CRA and/or NRA. In such instances, the gene encoding and expressing the CRA can be introduced to the target cell on a separate vector (preferably lacking the EBV-FR sequence and thus expressed or retained only transiently) than the NRS and the gene of interest. In such instances, the vectors respectively encoding the expression cassette(s) for the gene of interest or the CRA are preferably applied to the target cells or target tissues substantially simultaneously. As used herein the term substantially simultaneously shall mean that two, or more, compounds are introduced or otherwise applied to the body of the test animal or patient, or added to tissue or cells in culture, within 24 hours or each other, preferably within about 30 minutes of each other, within about 15 minutes, more preferably within about 5 minutes, specifically within about 1 minute, and most preferably simultaneously. The substantially simultaneous introduction of a vector encoding the gene of interest with a separate vector that provides relatively short term transient expression of the CRA or NRA is particularly useful in those instances where long term of expression of the CRA or NRA is deleterious to the cell.

ii. Expression Cassettes for Genes of Interest

For the purposes of the present invention, the “gene of interest” shall generally refer to any recombinantly encoded sequence that is not normally expressed in the target cells, or is normally expressed at levels substantially less (e.g., a least about 3 fold lower) than that obtained after a cell is treated with the presently described methods of gene delivery. The gene of interest can also be a replacement sequence targeted to a particular genomic locus for gene activation, repair or substitution purposes using homologous recombination. Examples of the specific sequences that can serve as the gene of interest include, but are not limited to, sequences encoding cytokines and growth factors, (such as GM-CSF, nerve growth factor (NGF), ciliary neurotropic factor (CNTF), brain-derived neurotropic factor (BDNF), interleukins 1-2 and 4-6, tumor necrosis factor-α(TNF-α), α or γ interferons, erythropoietin, and the like), the cystic fibrosis transmembrane conductance regulator (CFTR), tyrosine hydroxylase (TH), D-amino acid decarboxylase (DD), GTP cyclohydrolase (GTP) which can be delivered with or without TH and DD to treat, for example, Parkinson's disease by increasing L-dopa levels, leptin, leptin receptor, factors VIII and IX, tissue plasminogen activator (tPA), G-CSF, epo, selecting, adherins, integrins, proteoglycans, CRAs, NRAs and the like.

As used herein, the term “expression” refers to the transcription of the DNA of interest, and, optionally, the splicing, processing, stability, and translation of the corresponding MRNA transcript. Depending on the structure of the DNA molecule delivered, expression can be transient, intermittent, or continuous. “Durable” or “sustained” expression refers to the enhanced duration of the transient expression of the gene of interest that is afforded by the presence of the NRS or CRS in the described vectors (in conjunction with an appropriate exogenously added or endogenous CRA or NRA). Preferably, such vectors do not disrupt the structure of the host cell chromosomes via integration. Thus, durably transfected cells can be distinguished from cells that have been stably transduced by vectors that have integrated into the host cell chromosome.

An “expression cassette” includes both the gene of interest and at least one enhancer/promoter region that mediates the expression of the gene of interest which has been operably positioned proximal to the gene of interest. Given that gene expression can be linked to copy number, an additional embodiment of the presently described vectors are vectors incorporating multiple copies of the gene of interest, or multiple copies of expression cassettes containing the gene of interest. Where multiple copies of an expression cassette are present on a given vector, they can be situated either in the same or opposite orientation within the vector, can be located side-by-side, or can be interspersed throughout the vector with spacing regions of noncoding sequence of at least about 200-2000 bases. Typically, the number of duplicated expression cassettes or genes of interest within a typical vector shall be between about 2 and about 100. More typically between about 3 and about 60, and preferably between about 3 and about 20.

A number of transcriptional promoters and enhancers can be used to express the gene of interest, including, but not limited to, the herpes simplex thymidine kinase promoter, cytomegalovirus enhancer/promoter, SV40 promoters, and retroviral long terminal repeat (LTR) enhancer/promoters, hormone response elements, including GREs, AP-1, SP-1, Ets, NF-1, CREBs, or NFk-B binding DNA sequences and the like, as well as any permutations and variations thereof, which can be produced using well established molecular biology techniques (see generally, Sambrook et al. (1989) Molecular Cloning Vols. I-III, Cold Spring Harbor Laboratory Press, Cold Spring Harbor, N.Y., and Current Protocols in Molecular Biology (1989) John Wiley & Sons, all Vols. and periodic updates thereof, herein incorporated by reference). Enhancer/promoter regions can also be selected to provide tissue-specific expression, including expression targeted to vascular endothelial cells, monocytes, macrophages, lymphocytes, various progenitor and stem cells, such as hematopoietic stem cells, and the like. For purposes of the invention, enhancer/promoter elements are not simply multimers of consensus sequences known but are intact, preferably optimized, promoters linked to enhancer sequences.

RNAs of interest that can be delivered using the presently described methods include self-replicating RNAs, mRNA transcripts corresponding to any of the above genes which can be directly translated in the cytoplasm, or catalytic RNAs, e.g. “hammerheads” hairpins, hepatitis delta virus, group I introns which can specifically target and/or cleave specific RNA sequences in vivo. Of particular interest for targeting by catalytic RNAs are RNA viruses, as well as any of a wide variety of cellular or viral transcripts.

Alternatively, antisense forms of RNA, DNA, or a mixture of both can be delivered to cells to inhibit the expression of a particular gene of interest in the cell.

Additionally, the presently described vectors can incorporate features such as, but not limited to, multiple (one or more) expression cassettes, preferably from two to about 100 or more cassettes that each contain one or more enhancer/promoter elements, cDNAs and/or genomic clones, and polyadenylation sequences. One or more of the cassettes can also contain CRAs and/or NRAs. Each vector can also be engineered to contain specific intervening sequences between each expression cassette. These intervening sequences can vary from 10 to 5,000 bp in length, and can also contain sequences encoding a CRA or NRA, transcriptional enhancer or repressor sequences, nuclear localization and anchoring sequences from SV40 or other DNA sequences, and the like. Additionally, each vector can be engineered to contain cDNAs encoding transcriptional and/or post transcriptional enhancer elements or transcription factors, such as, but not limited to, AP-1, Sp-1, Nfkβ, ETS-1 or 2, NF-1, etc. Such factors can induce generalized, or alternatively tissue- and cell type-specific up- or down-regulation of transcription or post-transcriptional events. Additionally, enhancer or suppressor sequences can be included that specifically bind to and modulate gene expression from specific elements contained in the enhancer/promoter components of the expression cassette or intronic sequences within the genomic clones. Such sequences can include, for example, the HCMV IE1 and/or IE2 cDNAs to modulate the level of gene expression produced from the HCMV enhancer/promoter elements contained in the expression cassette. The present vectors can also be engineered to include inducible sequences, such as hormone response elements, including GREs, and or retinoic acid response elements that can be engineered either within the expression cassette itself or preferably upstream of the enhancer/promoter element and/or within the intervening sequences.

Yet another aspect of the expression cassettes for use in the vectors of the invention are those incorporating multiple enhancer/promoter elements operatively linked to the gene of interest. For example, two or more tandem enhancer/promoter elements are positioned upstream of the gene of interest. Surprisingly, these enhancer/promoter elements can function to enhance gene expression in either the 5′-3′ or 3′-5 direction, as long as at least one of the enhancer/promoter elements is positioned in the correct orientation upstream of the gene of interest (preferably the first or second most proximal enhancer/promoter element to the gene of interest).

Still another aspect of the invention are tissue specific enhancer/promoter elements. While generally tissue specific enhancer/promoter elements are weakly expressing, addition of multiple enhancer/promoter elements adjacent to the operatively linked enhancer/promoter element can further increase gene expression while maintaining tissue specificity.

Additional embodiments of the present invention include vectors encoding sequences that prevent the host cell from silencing vector encoded gene expression via, for example, methylation, rearrangement, deletion, or direct suppression. Examples of such sequences can include, but are not limited to, the presently described nuclear retention sequences, cellular retention sequences, and the cellular and nuclear retention activities.

An additional embodiment of the present invention includes vectors that have been packaged in conjunction with nuclear targeting peptides, or fusion proteins comprising specific or non-specific DNA binding activities, cellular retention and nuclear targeting domains. Examples of suitable DNA binding activities include, but are not limited to, the p53 binding domain, histone proteins, the glucocorticoid response element binding domain, the nonspecific DNA binding domain of a retroviral integrase protein, or the EBNA-1 protein (Middleton and Sugden, 1994, supra.). Particularly preferred embodiments of such domains include the DNA binding and/or nuclear retention domains of the EBNA-1 protein. These regions, which can be located in the N-terminal half, the C-terminal half, or the middle one third of the EBNA-1 coding region, or any substantially contiguous stretch of about 10 to about 100 amino acids, or preferably about 20 to about 60 amino acids therefrom, are preferred because such truncated forms of the molecule will preferentially delete the domain of the EBNA-1 protein that mediates the malignant transformation of mammalian cells in vitro. For example, the nuclear localization domain (NL) of EBNA-1 is located between about amino acid 379 through about amino acid 387, the dimerization and DNA binding domains of EBNA-1 are located between about amino acids 451 and about 604. Minimally, a variant EBNA-1 protein for use in the present invention will encode at least one or more of these regions while incorporating deletion (especially 20 to about 100 base deletions, or more, in the region encoding amino acids 89 through about 328), frameshift, or point mutations, or any combination of mixtures thereof in sequence encoding the N terminal 378 amino acids of the EBNA-1 protein. Examples of suitable point mutations include, but are in no way limited to, conservative amino acid substitutions, as well as substitutions designed to destroy an active sight such as exchanging phe and tyr residues, ser and thr residues (or either with ala, val, leu, etc.), asp and gly residues (or either with asn or gln), or replacing a cys with nonsulphur containing amino acid, at any one, several, or all position(s) where a given amino acid normally occurs in the EBNA-1 coding region. Such mutated EBNA-1 proteins are preferably substantially nontransforming. Of course, any of these nuclear targeting domains that can be packaged with the polynucleotide complexes can also be encoded by the nucleotides as part of the desired CRAINRA.

Examples of suitable nuclear targeting domains to be fused to the above DNA binding domains, or to be otherwise incorporated into the presently described polynucleotide complexes, include, but are not limited to, the nuclear localization sequences from: SV40 T antigen, histone amino acid sequences (especially the carboxy terminal domain), Qip 1, nuclear ribonucleoprotein A1 (especially the M9 domain), nuclear protein import factor p97 (especially the C-terminal 60% of the protein), the retinoblastoma tumor suppressor (especially amino acids 860-877 of the human retinoblastoma tumor suppressor), nucleoplasmin, c-Myc, or the CMV p65 lower matrix phosphoprotein.

iii. Other Aspects of the Vectors of the Invention

In addition to the specifically disclosed vectors, the presently described polynucleotide complexes can also be formed using a wide range of expression vectors including, but not limited to, a plasmid, a cosmid, a YAC, a BAC, a P-1 or related vector to optimally accommodate the gene of interest, a mammalian artificial chromosome, and a human artificial chromosome (HAC). Moreover, given the large size of the polynucleotide inserts supported by the present expression vectors, any of the aforementioned expression vectors (i.e., YACs, HACs, BACs, P1, cosmids, etc.), or any multiples or mixtures thereof, can be physically incorporated into the described vectors.

Given that non-viral methods of gene delivery are not constrained by the DNA packaging limitations inherent in viral based gene delivery methods, one embodiment of the present invention is a large vector. Typically, a large vector will have at least about 18 kb of recombinant genetic material, more typically at least about 20 kb of recombinant genetic material, preferably at least about 25 kb of recombinant genetic material up to about 1,000 kb. Typically, the genetic material is either RNA or DNA, and preferably DNA, and can comprise a proportion of nuclease resistant modified bases or chemical linkages.

Examples of such modified polynucleotides include, but are not limited to, those incorporating phosphorothioate linkages, 2′-O-methylphosphodiesters, p-ethoxy nucleotides, p-isopropyl nucleotides, phosphoramidites, chimeric linkages, and any other backbone modifications which render the polynucleotides substantially resistant to endogenous nuclease activity. Additional methods of rendering an polynucleotide nuclease resistant include, but are not limited to, covalently modifying the purine or pyrimidine bases in the polynucleotide. For example, bases can be methylated, hydroxymethylated, or otherwise substituted (glycosylated) such that polynucleotides comprising the modified bases are rendered substantially nuclease resistant.

Additionally, polynucleotides can be rendered substantially nuclease resistant by complexing the polynucleotides with any of a variety of packaging agents such as lipid emulsions, microcarrier beads, polymeric substances, proteins (preferably basic proteins), and the like.

Generally, a substantially nuclease resistant polynucleotide will be at least about 25% more resistant to nuclease degradation than an unmodified polynucleotide with a corresponding sequence, typically at least about 50% more resistant, preferably about 75% more resistant, and more preferably at least about an order of magnitude more resistant after 15 minutes of nuclease (e.g., human DNase) exposure.

Additionally, the vector can be linear but is preferably a covalently closed circle. Generally, the circle will be positively or negatively supercoiled, but, as in the case of nicked circles, can optionally have a relaxed topology.

When desired, the vectors can further incorporate a suicide signal that allows for the controlled extermination of cells harboring and expressing the gene of interest. For instance, the thymidine kinase (tk) gene can be incorporated into the vector which would allow a practitioner to subsequently kill cells expressing the tk gene by administering the correct amount of acyclovir, gangcyclovir, or the conceptual or functional equivalents thereof.

iv. Delivery and Expression

Given the presently described vectors' ability to express a gene of interest for prolonged periods, it is clear that the vectors are ideal for gene delivery applications both in vitro and in vivo. Accordingly, an additional aspect of the present invention is the use of the presently described vector/expression system to deliver genes of interest to suitable animal cells by any of a wide variety of techniques (see generally, Sambrook et al. (1989) Molecular Cloning Vols. I-III, Cold Spring Harbor Laboratory Press, Cold Spring Harbor, N.Y., and Current Protocols in Molecular Biology (1989) John Wiley & Sons, all Vols. and periodic updates thereof, herein incorporated by reference). Of particular interest are CLDC mediated gene delivery, viral gene delivery, polymer-based gene delivery, electroporation, nanoparticle/microcarrier bead mediated gene delivery, antibody conjugated DNA complexes, chemical transfection, delivery using complexed and naked forms of modified and/or unmodified polynucleotides, and the like.

Typically, expression using non-replicating and/or nonretained forms of the described vectors is transient: however, there are many instances where transient expression of recombinant genetic material of interest is more desirable. For example, transient expression can be preferred where one is simply delivering a viral receptor to the target cells in order the increase or enhance the infectivity of transducing virus that will integrate and stably express a cloned genetic material of interest (e.g., retrovirus or adeno-associated virus).

Additionally, transient expression is particularly preferable where acute diseases are involved. For example, cells can be temporarily rendered immune to specific antibiotic or chemotherapeutic agents by the introduction of a drug resistance factor. Since many cell populations are often adversely impacted by the effects of chemotherapeutic treatment, such cells can be transduced to transiently express factors that enhance the cells', and surrounding cells', resistance to a given treatment. Moreover, the presently described methods of transiently expressing the EBNA-1 gene can mediate durable expression of codelivered plasmids containing FR or oriP while avoiding or ameliorating the adverse consequences of long term, or durable, EBNA-1 expression.

Given the enhanced expression provided by the presently described vectors, “naked” forms of the vector can, for example, be directly injected into muscle where muscle cells take up and express the various gene products encoded by the vectors. Accordingly, “naked” DNA can act as a vaccine. Additionally, naked DNA can be incorporated into or onto any of a wide variety of implantable substrates including collagen supports, vascular grafts, stents, bone substitutes or cements, cartilage, biocompatible polymers and plastics, tendons and ligaments, and the like in order to allow host cells to take up the DNA and transiently express factors that enhance engraftment, or provide a particularly desirable therapeutic benefits. An additional application of such technology includes coating various surgical instruments (e.g., angioplasty balloons) with suitable DNA formulations in order to prevent or reduce complications such as restinosis.

The presently described vectors/expression system has also been introduced in vivo as naked DNA (without being packaged into conventional delivery vehicles such as virus, liposomes, or other ligand directed delivery vehicles, etc.) by mixing the purified plasmid DNA with agents such as calcium chloride, glycerol and other diluents and/or organic molecules, dextran 40 or other polymers, and glycoproteins (e.g., albumin, beta-2-microtubilin, lipoproteins, and particularly high density lipoprotein, and other serum proteins). A glycoprotein is a protein that contains some covalent glycosylation moiety. A purified glycoprotein is defined as at least a protein fraction that contains at least 90% of the desired glycoprotein(s), and more preferably 95%. For example, fractions of purified HDLs can be prepared by any of a number of methods known in the art such as that described by Fielding et al., 1986, J. Biol. Chem. 261:5233-5236. When naked DNA forms of the described vectors were systemically delivered using these formulations, significant levels of expression of the reporter gene were observed relative to vector only controls. Subsequent studies have shown that the expression of “naked” DNA can be further enhanced by adding additional agents to the DNA mixture, as well as treating the host animal before or after vector introduction with, for example, glucocorticoids, agents that promote endocytosis or stimulate cellular metabolism, sugars (e.g., mannitol), and lysosomal inhibitors. Similar methodologies are also suitable for localized gene delivery.

Optionally, the polynucleotide vectors can be condensed using suitable cations or cationic polymers prior to or during formulation for in vivo delivery.

The above polynucleotides can also be formulated in conjunction with polymer DNA complexes (see Goula et al, 1998, supra.), antibody conjugated polylysine-DNA complexes and other non-viral, non-lipid based DNA conjugate system as well as with naked DNA itself. Moreover, conventional modes of viral gene delivery can benefit by the incorporation of the presently disclosed NRS/NRA or CRS/CRA systems. Additional vectors that can be delivered using the presently disclosed methods and compositions include, but are not limited to, herpes simplex virus vectors, adenovirus vectors, adeno-associated virus vectors, retroviral vectors, lentivirus vectors, pseudorabies virus, alpha-herpes virus vectors, and the like. A thorough review of viral vectors, particularly viral vectors suitable for modifying nonreplicating cells, and how to use such vectors in conjunction with the expression of polynucleotides of interest can be found in the book Viral Vectors: Gene Therapy and Neuroscience Applications Ed. Caplitt and Loewy, Academic Press, San Diego (1995). Additionally, the presently described methods can be used to complex and deliver viral or subviral particles encoding or containing the genetic material of interest.

The presently described lipid and polymer complexes are specifically designed to deliver genetic material of interest to cell or tissues in vivo. Consequently, it is important that the materials to be incorporated into the described complexes, or used during the formulation of the complexes, have a low inherent toxicity. For example, the various biochemical components of the present invention are preferably of high purity and are substantially free of potentially harmful contaminants (e.g., at least National Food (NF) grade, generally at least analytical grade, and preferably at least pharmaceutical grade). To the extent that a given compound must be synthesized prior to use, such synthesis or subsequent purification shall preferably result in a product that is substantially free of any potentially toxic agents which can have been used during the synthesis or purification procedures. Additionally, the pre-treatment of the gene delivery recipient with, for example, dexamethasone or other corticosteroids can also reduce host toxicity.

Additionally, the polynucleotides to be delivered should be substantially pure (i.e., substantially free of contaminating proteins, lipid, polysaccharide, lipopolysaccharide, nucleic acid, and potentially CpG sequences that can be immunogenic). Where plasmid DNA is used, the preparations will generally be prepared by a process comprising phenol, or phenol:chloroform, extraction, and isopycnic centrifugation (using CsCl, and the like), or functional equivalents thereof. Preferably, the DNA preparations will also be treated with RNase, and subject to multiple rounds of extraction with organic solvents, and at least two rounds of ultracentrifugation (or any other means of producing DNA at least as pure). Typically, a substantially pure preparation of nucleic acid is a preparation in which at least about eighty percent, generally at least about ninety percent, and preferably at least about ninety five percent of the total nucleic acid is comprised of the desired nucleic acid.

Additionally, many commercially available acyl chain cationic lipids are relatively toxic to target cells and tissues. Consequently, such compounds are not preferred for the practice of the claimed invention. Of particular interest are cationic lipids used in conjunction with cholesterol. Such compounds, particularly dimethyl dioctadecyl ammonium bromide (DDAB) or DOTIM, preferably used 1:1 with cholesterol, can be formulated with polynucleotides to yield a complex with a relatively low in vivo toxicity. As such, cholesterol groups that have been suitably mixed with, or derivatized to, cationic groups are particularly well suited for the practice of the presently described invention.

The cationic component of a suitable cholesterol lipid can comprise any of a variety of chemical groups that retain a positive charge between pH 5 through pH 8 including, but not limited to, amino groups (or oligo or poly amines), e.g., spermine, spermidine, pentaethylenehexamine (PEHA), diethylene triamine, pentamethylenehexamine, pentapropylenehexamine, etc.), amide groups, amidine groups, positively charged amino acids (e.g., lysine, arginine, and histidine), imidazole groups, guanidinium groups, or mixtures and derivatives thereof. Additionally, cationic polymers of any of the above groups (linked by polysaccharide or other chemical linkers) have also proven useful in gene delivery and can be incorporated into the presently described lipid complexes. The cross-linking agents used to prepare such polymers are preferably biocompatible or biotolerable, and will generally comprise at least two chemical groups (i.e., the cross-linkers are bifunctional) that are each capable of forming a bond with a suitable chemical group on the cation. For the purposes of the present disclosure, the term biocompatible shall mean that the compound does not display significant toxicity or adverse immunological effects at the contemplated dosages, and the term biotolerable shall mean that the adverse biological consequences associated with a given compound can be managed by the appropriate dosaging regimen or counter-therapy. The linker groups can be homobifunctional (same chemical groups) or heterobifunctional (different chemical groups). Optionally, in order to facilitate the release of the vector from the complex, the chemical linkage formed between the linking group and the cationic moiety will preferably be hydrolyzable under physiological conditions (i.e., pH labile, or otherwise subject to breakage in the target cell). Additionally, the cross-linking agent can comprise a bond that is hydrolyzable under physiological conditions in between the linking groups.

Optionally, the cross-linking agent can be combined with an additional cross-linking agent that a allows for the formation of branched polymers. By varying the ratio of the branching linking molecules to polymerizing cross-linker, cationic polymers are produced with a variety of chemical characteristics.

Where appropriate, any or a variety (i.e., mixture) of other “helper” lipid moieties can be added to the presently described lipid or polymer/polynucleotide delivery vehicles as necessary to provide complexes with the desired characteristics. As such, any of a number of well known phospholipids can be added including, but not limited to, disteroylphosphatidyl-glycerol (DSPG), hydrogenated soy, phosphatidyl choline, phosphatidylglycerol, phosphatidic acid, phosphatidylserine, phosphatidylinositol, phosphatidyl ethanolamine, sphingomyelin, mono-, di-, and triacylglycerols, ceramides, cerebrosides, phosphatidyl glycerol (HSPG), dioleoyl-phosphatidylcholine (DOPC), dilauroylphosphatidyl-ethanolamine (DLPE), cardiolipin, and the like. Typically, helper or otherwise neutral lipid shall comprise between about 15 percent to about 70 percent of the lipid component of a polynucleotide delivery complex, preferably between about 15 and about 60 percent, more preferably between about 30 and 60 percent, and more typically at least about 60 percent, and specifically at least about 50 percent. Conversely, the percentage of cationic lipid will preferably constitute about 30 to about 70 percent of the net lipid component of the complex, more preferably about 40 to about 60 percent, and specifically about 50 percent.

Viral based systems of gene delivery are generally constrained by the inherent immunogenicity of the virions used to effect gene delivery. Once a patient has been primed to respond to a given virus, neutralizing antibodies and cytotoxic T lymphocytes can hinder gene delivery using the virus, or antigenically related viruses. Consequently, an additional embodiment of the present invention includes non-viral lipid and/or polymer-polynucleotide complexes that are characterized by having low immunogenicity. For the purposes of the present disclosure, the term low immunogenicity shall mean that neutralizing titers of complex-specific antibodies or immunizing quantities of vector specific T lymphocytes are not found in the blood of a majority of immunocompetent patients after three or more in vivo applications of the complexes into patients. Alternatively, the term low immunogenicity can mean that titers of complex specific antibodies, or levels of complex specific immune T lymphocytes are generally at least about 50 percent less than titers observed after the i.v. or i.m. injection of at least about 10¹¹ replication defective adenovirus particles.

Additionally, the term non-viral shall refer to the fact that a given gene delivery complex or method does not incorporate a sufficient amount of viral capsid or envelope protein, or portions thereof, to stimulate a host immune response against the viral protein. Typically, the presently described non-viral methods of gene delivery (e.g., CLDC, polynucleotide complexes and/or polymers, etc.) can be used as a primary means of gene delivery in vitro or in vivo. However, this fact by no means precludes the use of the presently described non-viral gene delivery systems as a follow-up, or booster, gene delivery treatment subsequent to initial viral mediated gene delivery.

Additionally, the polynucleotide complexes can also be modified to enhance their in vivo stability as well as any of a variety of pharmacological properties (e.g., increase in vivo half-life, further reduce toxicity, etc.) by established methods. For instance, the polynucleotide complexes can be formulated to deliver polynucleotides to the body in a time-released manner or contain agents that prolong circulation time of circulating materials, such as polyethylene glycol. Such time release formulations are contemplated to facilitate the treatment of acute conditions by providing extended periods of transient gene delivery, or providing practitioners with alternative means of dosaging and delivering nucleic acid in vivo. In particular, the presently described complexes are ideal for the packaging and delivery of polynucleotide based vaccines. Vaccines of particular interest include nucleotides encoding toleragens, immunogens from both eucaryotic and procaryotic pathogens, viruses, and tumor associated antigens.

Where diagnostic, therapeutic or medicinal use of the presently described polynucleotide complexes is contemplated, the complexes can be prepared and maintained under sterile conditions in order to avoid microbial contamination. Because of the relatively small size and inherent stability of the complexes, they can also be sterile filtered prior to use. In addition to the above methods of sterile preparation and filter sterilization, antimicrobial agents can also be added. Antimicrobial agents which can be used, generally in amounts of up to about 3% w/v, preferably from about 0.5 to 2.5%, of the total formulation, include, but are not limited to, methylparaben, ethylparaben, propylparaben, butylparaben, phenol, dehydroacetic acid, phenylethyl alcohol, sodium benzoate, sorbic acid, thymol, thimerosal, sodium dihydroacetate, benzyl alcohol, cresol, p-chloro-m-cresol, chlorobutanol, phenylmercuric acetate, phenylmercuric borate, phenylmercuric nitrate and benzylalkonium chloride. Preferably, anti-microbial additives will either enhance the biochemical properties of the polynucleotide complexes, or will be inert with respect to complex activity.

Methods of preparing DNA:lipid complexes for in vivo gene delivery are generally described in Liu et al, 1995, J. Biol. Chem., 270(42):24864-24870 which is herein incorporated by reference. In brief, 360 μg of purified vector DNA in 600 μl of D5W was rapidly introduced to a tube containing cationic liposomes (DDAB, DOTIM, or DOTMA in a 1:1 ratio with cholesterol, 5.760 μmol of total lipid) and gently mixed.

During assembly, the cationic component will generally be combined with the polynucleotide at a cation/phosphate ratio that has been optimized for a given application. Usually, the DNA phosphate:cation ratio will be between about 1:8 (μg DNA:nmol cationic lipid), preferably between about 2:1 and about 1:16 for intravenous administration, and about 1:1 for i.p., or aerosol applications, and the like.

Since ion pairing plays a role in the formation of the cation/polynucleotide complexes, the pH during complex formation can be varied to optimize or stabilize the interaction of the specific components. For instance, where non-pH sensitive cationic lipids are used, a pH as low as about 5 can be preferred to complex a given polynucleotide (e.g., RNA) or other chemical agent which can be coincorporated with the polynucleotide. Additionally, where the polynucleotide (e.g., DNA) is not substantially sensitive to base hydrolysis, circumstances can dictate that a pH of up to about 10 be used during complex formation. Generally, a pH within the range of about 5 to about 9, and preferably about 7, will be maintained during complex formation and transfection.

Similarly, the concentration of salt (e.g., NaCl, KCl, MgCl₂, etc.) can be varied to optimize complex formation, or to enhance the efficiency of gene delivery and expression. Additionally, factors such as the temperature at which the cationic lipid is complexed to the polynucleotide can be varied to optimize the structural and functional attributes of the resulting complexes. Additionally, the osmolarity of solution in which the complexes are formed can be altered by adjusting salt or other diluent concentration.

Since moderate concentrations of salt can impede complex formation, one can also adjust osmolarity by adding or substituting suitable excipients such as, but not limited to, glucose, sucrose, lactose, fructose, trehalose, maltose, mannose, and the like. The amount of sugar (dextrose, sucrose, etc., see list provided above) that can be present during complex formation shall generally vary from between about 2 percent and about 15 percent, preferably between about 3 percent and about 8 percent, and more preferably about 5 percent.

Alternatively, the osmolarity of the solution can also be adjusted by a mixture of salt and sugar, or other diluents including dextran 40, albumin, serum, lipoproteins, and the like. One skilled in the art would clearly know how to appropriately vary the concentration of salt and sugar to optimize the efficiency of gene delivery. Typical concentrations of salt and sugar that can serve as a starting point for further optimization are about 250 mM (glucose) and about 25 mM salt (NaCl). An additional feature of complex formation is temperature regulation. Typically, cationic lipids are complexed with polynucleotide at a temperature between about 4° C. and about 65° C., more typically between about 10° C. and about 42° C., preferably between about 15° C. and about 37° C., and more preferably at about room temperature. In many instances, precise regulation of temperature during complex formation (e.g., +/−1° C.) is important to minimizing product variability.

Depending on the formulation used to produce the polynucleotide complexes of the present invention, the resulting complexes will typically vary in size and structure. For example, lipid complexes formed using DOTMA in conjunction with DOPE or cholesterol typically form small unilamellar vesicles (SUV) with diameters of between about 50 mn and about 100 nm. Lipid complexes formulated with DOTIM and prepared by hand shaking or vortexing typically produce multilamellar vesicles with varying diameters substantially larger than 200 nm.

Since the presently described lipid/polynucleotide or polymer/polynucleotide complexes can be formulated into stable vesicles having a particular range of sizes, targeting agents can be incorporated into vehicles to direct the vehicles to specific cells and/or tissues. Accordingly, any of a variety of targeting agents can be also be incorporated into the delivery vehicles.

For the purposes of the present disclosure, the term targeting agent shall refer to any and all ligands or ligand receptors which can be incorporated into the delivery vehicles. Such ligands can include, but are not limited to, antibodies such as IgM, IgG, IgA, IgD, and the like, or any portions or subsets thereof, cell factors, cell surface receptors such as, integrins, proteoglycans, sialic acid residues, etc., and ligands therefor, MHC or HLA markers, viral envelope proteins, peptides or small organic ligands, derivatives thereof, and the like.

The targeting ligand can be derivatized to an appropriate portion of the cationic polymer prior to the formation of the polynucleotide delivery vehicle. For example, the targeting agent (e.g., immunoglobulin) can be N-linked to a free carboxyl group of the polar region of a branched cross-linking molecule, by first derivatizing a leaving group to the carboxyl group using N-hydroxysuccinimide (NHS) and 1-ethyl-3-(3-dimethylaminopropyl)carbodiimide (EDAC), or the methiodide thereof, (EDC methiodide) and a free amino group on the targeting molecule. Alternatively, targeting agents can be disulfide linked to a properly conditioned linking agent or cation (using thioacetic acid, hydroxylamine, and EDTA).

Alternatively, the targeting agent can also act as a bridge between the polynucleotide complex and the “targeted” cells or tissues, For instance, where the targeting agent simply associates with the complex, the agent can be added to the complex well after complex formation or isolation. To the extent that the targeting agent is also capable of recognizing, or being recognized by, molecules on the cell surface, it can act as a bridge molecule which effectively places the complex in intimate contact with the cell surface.

Proteins that associate with the polynucleotide complexes can also be derivatized with a targeting ligand and used to direct complexes to specific cells and tissues. In this manner, any of a variety of cells such as endothelial cells, stem cells, germ line cells, epithelial cells, islets, neurons or neural tissue, mesothelial cells, osteocytes, chondrocytes, hematopoietic cells, immune cells, cells of the major glands or organs (e.g., lung, heart, stomach, pancreas, kidney, skin, etc.), exocrine and/or endocrine cells, and the like, can be targeted for gene delivery. Alternatively, any or all of the above cells or tissues can serve as targets for gene delivery using polynucleotide complexes that do not incorporate specific targeting ligands.

Of particular interest for targeted gene delivery applications similar to those outlined above are proteins encoding various cell surface markers and receptors. A brief list that is exemplary of such proteins includes, but is not limited to: CD1(a-c), CD4, CD8-11(a-c), CD15, CDw17, CD18, CD21-25, CD27, CD30-45(R(O, A, and B)), CD46-48, CDw49(b,d,f), CDw50, CD51, CD53-54, CDw60, CD61-64, CDw65, CD66-69, CDw70CD71, CD73-74, CDw75, CD76-77, LAMP-1 and LAMP-2, and the T-cell receptor, integrin receptors, endoglin for proliferative endothelium, or antibodies against the same.

Where a targeting agent has been assembled within the polynucleotide complex, a suitable ligand or antibody, or mixture thereof, can be affixed to a suitable solid support, i.e., latex beads, microcarrier beads, membranes or filters, and the like, and used to selectively bind and isolate complexes that incorporate the targeting receptor or ligand from the remainder of the preparation. Thus, a method is provided for isolating the desired polynucleotide complexes prior to use.

Additionally, any of a variety of chemical stabilizing agents can be utilized in conjunction with the described complexes. Suitable pharmaceutically acceptable antioxidants include propyl gallate, butylated hydroxyanisole, butylated hydroxytoluene, ascorbic acid or sodium ascorbate, DL- or D-alpha tocopherol and DL- or D-alpha-tocopherol acetate. The anti-oxidant, if present, can be added singly or in combination to the polynucleotide delivery vehicles either before, during, or after vehicle assembly in an amount of up to, for example, 0.1% (w/v), preferably from 0.0001 to 0.05%.

Cationic liposomes are typically stored at 4° C. under an inert gas or are lyophilized and reconstituted prior to complexation. DNA:lipid complexes can be lyophilized and reconstituted prior to use.

If desired, one or more stabilizers and/or plasticizers can be added to polynucleotide complexes for greater storage stability. Materials useful as stabilizers and/or plasticizers include simple carbohydrates including, but not limited to, glucose, galactose, sucrose, or lactose, dextrin, acacia, carboxypolymethylene and colloidal aluminum hydroxide. When stabilizers/plasticizers are added, they can be incorporated in amounts up to about 10% (w/v), preferably from about 0.5 to 6.5%, of the total preparation. Additionally, the presently described polynucleotide complexes can be stored frozen or as a lyophilized cake or powder.

b. Somatic Transgenic Non-Human Animals And Functional Genomics

An ever increasing fraction of the human genome, as well as the genomes of various animal species, is being sequenced, and a large number of coding regions are being identified within this sequenced DNA. However, the function of the protein or nucleic acid products of very large numbers of these sequenced coding regions remain unknown, and cannot be deduced from currently available approaches to functional genomics. In addition, large numbers of nucleic acid sequences coding for potentially important protein/nucleic acid products are being identified through the use of substraction hybridization techniques. These techniques can be used to isolate genes which are differentially expressed under a wide variety of biologic conditions in vitro as well as in vivo. Many of these differentially expressed genes can code for nucleic acids/protein products which perform important biologic function in intact hosts. However, many of these differentially expressed clones code for products for which no known function has been identified to date.

The presently disclosed methods and compositions are particularly well suited for the delivery of genes in vivo. Consequently, an additional feature of the present invention are non-human somatic cell transgenic animals that have been genetically altered to express a gene or genes of interest.

The use of somatic cell transgenic animals as described herein can revolutionize functional genomics. Specifically, the presently described methods and vectors provide the ability to express essentially any cDNAs or genomic clones at biologically significant levels for extended periods of time in animals. This feature of the presently described invention enables one to assess the (previously unknown) function of a given gene product to be identified in a somatic cell transgenic animal system. Additionally, the progression, amelioration, or prevention of disease states can be monitored using suitable genetically modified somatic cell transgenic animal models. Using this approach, a variety of parameters are monitored in the somatic cell transgenic animal, including appearance (skin, hair, etc.), full blood counts and blood chemistries, cytokine levels, full histopathologic analysis, including monitoring for possible organ changes of injury, inflammatory responses and/or the induction of disease states, including cancer, heart disease, atherosclerosis, hypertension, diabetes, asthma, maintenance of body weight, etc. Alternatively, this approach can be used to express genes whose function is unknown in animal models of cancer, heart disease, atherosclerosis, hypertension, diabetes, asthma, etc. in order to determine whether in vivo expression of one or more of these genes can produce significant therapeutic effects in animal models directly relevant to common human diseases. By correlating the observed phenotypic changes with the introduction of specific cDNAs (by comparing the treated animals with both mock-treated and untreated control animals) the specific function(s) of these uncharacterized DNA sequences can be assessed. This approach is of greatest utility for full length cDNAs or genomic clones, or for partial clones from which full length clones can be generated.

The somewhat transient nature inherent in the presently described transgenic animals also allows for the assessment of transient manipulations of the animal's genotype. This feature is particularly useful where one is studying the effects of exogenously added genes that are unduly toxic when stably and continuously expressed. Another feature of the present methods allows for the transient assessment of effects correlating with transient expression of the gene of interest as well as changes that occur in the test cells or animals as expression slowly diminishes. Finally, the presently described methods are ideally suited for assessing the transient effects of specifically inhibiting or reducing the expression of otherwise essential cellular genes. For example, the genes of interest in such vectors can encode antisense messages, targeted ribozymes, or inhibitory proteins or peptides, that disrupt the normal expression of a given cellular gene. By monitoring test animals and cells treated as described above, one can study the cellular and phenotypic effects of selectively inhibiting the expression of virtually any gene in the cell. Accordingly, a key aspect of the present invention is that a method is provided for identifying those genes involved in a given regulatory pathway by serially testing which genes are affected by the targeted reduction or augmentation of the expression of a given cellular gene.

Another strength of the somatic cell transgenic approach for functional genomics is that very large DNA vectors can be delivered and efficiently expressed in animals using this approach. Therefore, five to ten or more different DNA sequences of unknown function can be incorporated into a single vector and expressed in a single animal. This approach dramatically increases the number of unknown DNA coding regions that can be assessed at one time, and makes this approach more economically feasible. Furthermore, the animals can be made transgenic by injection of the genes of interest systemically, into the central nervous system, into a specific tissue or into growing fetuses in utero, in order to maximize the ability to identify genes that have novel functions in the CNS or during early development, as widespread systemic functions.

Typically, the presently described method for practicing functional genomics will express the genes of interest at biologically and therapeutically relevant levels for prolonged periods without producing significant ongoing host toxicity and without producing a phenotype based on host-immune, toxic, or transforming responses. Additionally, the present methods allow for the efficient re-expression the gene(s) of interest after reinjection into immunocompetent hosts. Thus, expression can be maintained for very long periods if such periods are required in order to induce a phenotype. Also, the present methods allow the delivery and expression of very large DNA vectors, which allows the delivery and expression of multiple different cDNAs and/or genomic clones into a single animal. In this way, potential in vivo interactions of two or more genes can be readily assessed. Moreover, large numbers of unknown genes can be expressed in a single animal, thus allowing the functional screening of very large numbers of unknown genes using relatively few animals. This feature of the presently described methods substantially increases the efficiency of screening, and significantly reduces the numbers of animals required to screen large numbers of genes. The present method can also be used efficiently via a variety of routes of administration, including systemic, directly into the CNS and directly into developing fetuses in utero. Thus, the function of unknown genes can be assessed in multiple tissues, as well as in utero and during early post-natal development. This maximizes the likelihood of identifying the function of unknown genes in the maximal number of tissues and cellular sites during the different stages of development from fetal life through adulthood.

Using this methodology, one can rapidly determine, or gain insight into, the function of a cloned gene or cDNA sequence in test animals. Accordingly, the presently described methods and technology are particularly well suited for the practice of functional genomics. Such in vivo functional genomic studies would particularly benefit from the prolonged but still transient nature of gene expression inherent in a subset of the methods herein described. For example, regulatable expression of engineered genes in mammalian cells remains an elusive goal of genetic researchers. Using the present system, a gene of interest can be “induced” by simply injecting a suitable polynucleotide complex into a living test animal, gene expression can be maintained by subsequent treatments, and gene expression can be terminated by the cessation of further treatments. In essence, the present system describes a effective system for the regulatable expression of test genes in living animals. As such, the potential application of the present technology for functional genomic testing, or even treatment for acute medical conditions, are evident to those skilled in the art. It is also possible to use the presently described methods to generate somatic cells transgenic animals that are used to produce relatively large quantities of recombinantly encoded products such as, for example, human factor VIII, factor IX, etc.

In addition to the specifically exemplified mice, examples of mammalian species that can be used in the practice of the present invention include, but are not limited to: humans, non-human primates (such as chimpanzees), pigs, rats (or other rodents), rabbits, cattle, goats, sheep, and guinea pigs. Additionally, as non-viral methods of gene delivery are not limited to specific species or animal types, the presently described methods are also suitable for use in the production of non-mammalian somatic cell transgenic animals such as insects, arthropods, crustaceans, birds, and fish.

The presently described methods for gene delivery are also well suited for practicing functional genomics in vitro and in vivo. For example, gene expression profiles can be determined for cells or animals that have been transiently transfected to durably express a gene of interest and compared to the expression profiles for normal and mock transfected cells. As the gene gradually disappears from the cell population, the changes in the gene expression profile can be monitored to develop a highly refined understanding of the functionality of the delivered gene. Optionally, a similar methodology can be used to test different combinations of genes, and combinations of genes that have been introduced to cells or animals in a specific order. Additionally, the presently described methods can be used to delivery marker or test genes into a population of cells that have a well characterized and understood genetic background without disrupting the cellular genome. Accordingly, the present methods and vectors are particularly well suited to the delivery of both test and marker genes to cells for use in high-throughput screening assays. Examples of such an assays can be found, inter alia, in U.S. Pat. Nos. 5,491,084 and 5,625,048, both of which are herein incorporated by reference. In addition to the above-identified genes, genes encoding G proteins, promiscuous G proteins, beta-lactamases and derivatives thereof, green fluorescent protein and derivatives thereof, cell surface receptors, cell membrane proteins, intercellular and intracellular signal transduction proteins, oncogenic proteins, mitogenic proteins, DNA repair proteins, cytoskeletal proteins, and the like, can be introduced to target cells using the described vectors and methods.

Preferably, cells transduced using the presently described vectors and methods remain suitable for screening of combinatorial libraries of proteins, nucleotides, and small organic molecules. Moreover, the present methods are also compatible with screening of combinatorially produced or other test compounds that are added to cells before, simultaneously with, and after the introduction of a test gene, or genes.

Virtually any cell type from any animal can be used in the above screening assays as long as the cell is capable of internalizing and expressing the presently described recombinant vectors. Optionally, the target cells can be transduced to express or over express proteoglycan, or other, receptors that mediate or facilitate the uptake of the presently described vectors or polynucleotide complexes. Cell types that are particularly preferred include, but are not limited to, liver cells (hepatocytes), lung cells, blood cells, stem cells, fibroblasts, white blood cells, endothelial cells, macrophages, monocytes, dendritic cells, neural cells, astrocytes, muscle cells, and the like.

In brief, the presently described invention represents a new and powerful approach to functional genomics using somatic cell gene delivery in animals. This non-viral, non-germline-based in vivo gene delivery approach can be used to identify an unknown function, or study the known function of essentially any gene product (either RNA or proteins) in intact living organisms. The determination of gene function in living animals for the first time permits the identification of large numbers of new disease-causing or associated genes, as well as novel genes whose in vivo transfer and expression produces therapeutic gene products for the treatment of human and veterinary diseases.

Prior to the present invention, CLDC-based in vivo gene delivery has not been able to identify the functional activities of genes and/or cDNAs for which function has not yet been identified, nor has it been used to identify novel and unanticipated functions for genes/cDNAs for which limited functionality has already been identified. The present invention, for the first time, permits the use of non-viral gene delivery in order to identify gene function in animals. Consequently, the present invention describes the first use of non-viral gene delivery, including CLDC-based in vivo gene delivery, to identify gene function in animals. During these studies, the following breakthroughs were required/made:

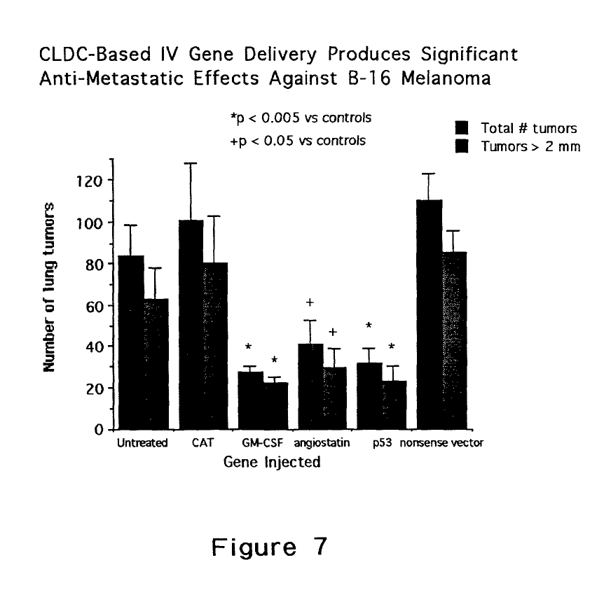

A) A novel plasmid-based expression system that produces both long-term expression of delivered genes or cDNAs at biologically and therapeutically significant levels following administration directly into animals, and efficient re-expression following re-injection into immunocompetent animals (see FIG. 8).

B) Demonstration, for the first time, that CLDC-based gene delivery into tumor bearing mice can identify novel and unexpected anti-tumor functions for genes for which another, completely unrelated anti-tumor function has already been identified.

C) In vivo administration of multiple genes simultaneously in order to determine whether one or more individual genes of unknown function, co-delivered with a gene/cDNA of known function produces i) additive or synergistic activity with the known function, ii) inhibits the known function, or iii) has no effect on that function, and thus to identify the function of the unknown gene by loss or gain of function determination.

D) A single expression plasmid that contains multiple expression cassettes and that can efficiently express multiple different genes simultaneously, for prolonged periods, at biologically and therapeutically significant levels.

Although the use of the presently described vectors and methods are preferred, the use of CLDC (or any other nonviral method of gene delivery) to assess gene function in vivo and to identify biological, biochemical, and genetic pathways in vivo is not deemed to be limited to the specifically described vectors. Given the present teaching, any of a wide variety of suitably constructed vectors can be used to practice functional genomics in conjunction with one or more of the described methods of CLDC formulation and delivery, host pretreatment, etc.

For example, as proof of principle, the presently described invention has shown that absolute neutrophilia, an important in vivo phenotype produced by the transfer and expression of the human granulocyte colony-stimulating factor (hG-CSF) gene (Petros, 1992, Pharmacotherapy, 12:32S-38S), can be identified in mice using CLDC-based in vivo gene delivery only if the vector system of the invention is used to deliver the hG-CSF gene. The use of CLDC incorporating p4305 (a conventional hG-CSF expression plasmidpreviously considered to be state of the art (Y. Liu et al., 1997, Nature Biotechnology: 15:167-173) produced high levels of hG-CSF gene expression transiently in injected mice, but did not produce sufficiently long-term expression of hG-CSF to elevate neutrophil counts at any timepoint after i.v. injection of CLDC (see Table 2 below).

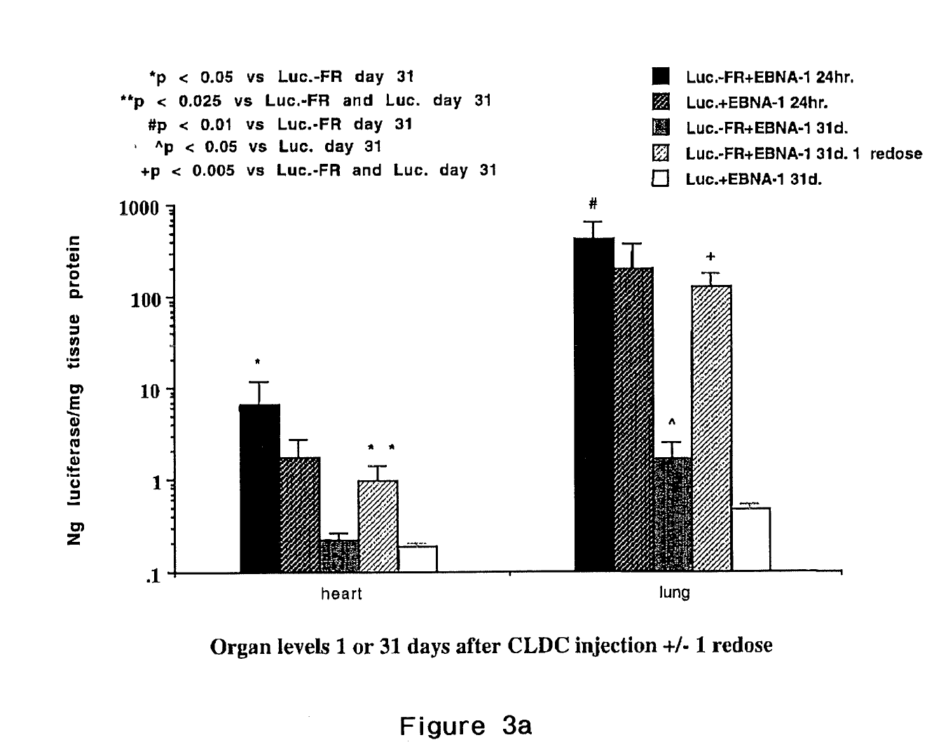

Furthermore, the presently described EBV-based two plasmid system can be used to efficiently re-express the delivered genes following re-injection of the CLDC into immunocompetent animals (FIG. 3). Accordingly, the present invention permits the identification of genes that require either long term or chronic expression to manifest a phenotype in vivo. Without the use of the present EBV-based vector system model, it would not be possible to identify phenotypes for very large numbers of genes (including the hG-CSF gene, other CSFs, growth factors, etc.) following non-viral gene delivery into mice.

C. Gene Therapy

Another embodiment of the subject invention involves the use of the presently described methods and compositions to effect gene therapy. Such gene therapy is intended to compensate for genetic deficiencies in the afflicted individual's genome and can be effected by ex vivo somatic cell gene therapy whereby host cells are removed from the body are transduced to express the deficient gene and reimplanted into the host. Alternatively, somatic cell gene therapy can be effected by directly injecting a vector bearing the desired gene into the individual, in vivo, whereby the gene will be delivered and expressed by host tissue. In the presently described instance, the vector shall preferably be introduced to target cells substantially simultaneously with polynucleotide sequence encoding a cellular retention activity and/or a nuclear retention activity.

The presently described polynucleotide complexes can be introduced in vivo by any of a variety of established methods. For instance, they can be administered by inhalation, by subcutaneous (sub-q), intravenous (I.V.), intraperitoneal (I.P.), intracranial, intraventricular, intrathecal, or intramuscular (I.M.) injection, rectally, as a topically applied agent (transdermal patch, ointments, creams, salves, eye drops, and the like), or directly injected into tissue such as tumors or other organs, or in or around the viscera.

Since the presently described methods and compositions are suited for the delivery of genes to both normal cells and tumor cells, an additional embodiment of the present invention is the use of the disclosed methods to deliver genes encoding antitumor agents to patients. For example, immune stimulants, tumor suppressor genes, or genes that hinder the growth, local extension, or metastatic spread of tumor cells can be delivered to tumor cells and other target cells, including, but not limited to, vascular endothelial cells and immune effector and regulator cells that subsequently express the genes to the detriment of the tumor. Particular examples of such genes include, but are not limited to: angiostatin, p53, GM-CSF, IL-2, G-CSF, BRCA1, BRCA2, RAD51, endostatin (O'Reilly et al., 1997, Cell, 88(2):277-285), TIMP 1, TIMP-2, Bcl-2, and BAX. Furthermore, similar methodologies can be employed to generate cancer vaccines similar to those disclosed in U.S. Pat. No. 5,637,483, issue to Dranoff et al., herein incorporated by reference. In view of the above, the presently disclosed methods and compositions are also useful for the treatment of cancer. Cancers that can be prevented or treated by the methods of the invention include, but are not limited to: cardiac; lung; gastrointestinal; genitourinary tract; liver; bone; nervous system; gynecological; hematologic; skin; and adrenal glands. The present vectors and methods are also suitable for therapeutic or preventative treatment of the normal tissues from which the such cancers originate.

Given the contemplated in vivo use of the described lipid/polynucleotide complexes, it is important that, to the extent possible, all components and materials used during the assembly and production of the lipid/polynucleotide complexes be biocompatible or biotolerable. As such, the lipid complexes of the present invention are preferably formed using solutions and compounds that are routinely used in the treatment of patients. Examples of such solutions and compounds include, but are not limited to: Ringers lactate, 5 percent dextrose, buffered saline, dextran 40, serum proteins, protamine sulfate, albumin, purified lipoproteins or lipoprotein fragments, human serum, transferrin, albumin, or mixtures thereof.

One of ordinary skill will appreciate that, from a medical practitioner's or patient's perspective, virtually any alleviation or prevention of an undesirable symptom (e.g., symptoms related to disease, sensitivity to environmental factors, normal aging, and the like) would be desirable. Thus, for the purposes of this Application, the terms “therapy”, “treatment”, “preventative treatment”, “therapeutic use”, or “medicinal use” used herein shall refer to any and all uses of the claimed compositions which remedy a disease state or symptoms, or otherwise prevent, hinder, retard, or reverse the progression of disease or other undesirable symptoms in any way whatsoever.

When used in the therapeutic or preventative treatment of disease, an appropriate dosage of polynucleotide delivery complex, or a derivative thereof, can be determined by any of several well established methodologies. For instance, animal studies are commonly used to determine the maximal tolerable dose, or MTD, of bioactive agent per kilogram weight. In general, at least one of the animal species tested is mammalian. Those skilled in the art regularly extrapolate doses for efficacy and avoiding toxicity to other species, including human. Before human studies of efficacy are undertaken, Phase I clinical studies in normal subjects help establish safe doses.

d. Host Specific Factors In Gene Delivery

In vivo gene delivery studies have shown that different strains of a given species can display widely varying efficiencies of gene transfer and expression. For example, the present disclosure describes non-viral gene delivery experiments demonstrating that Swiss Webster mice display different levels of expression as compared to ICR mice. Similarly, the present disclosure reveals that FVB mice display significantly different efficiencies of gene delivery. Accordingly, host physiology can apparently play an important role in the efficiency of gene delivery. As such, another aspect of the present invention is a method of in vivo gene delivery that involves the treatment of patients with an agent before, concurrently with, or after gene delivery. One example of such an agent is dexamethasone. Additional agents include, but are not limited to: corticosteroids, or the formulation of cationic liposome:DNA complexes in diluents including dextran 40, lactated ringers, albumin, protamine sulfate, and/or serum and the like.

The presently described studies indicate that the cationic moiety of a lipid/polynucleotide complex binds to membrane associated proteoglycans and that proteoglycans are essential for cationic lipid-mediated gene delivery in vivo. Accordingly, one can modulate gene delivery by modulating the levels of proteoglycans present on the surface of the cell.

Additionally, as membrane associated proteoglycans have been implicated in the cellular uptake of all cationic vehicle polynucleotide complexes, polynucleotide complexes incorporating ligands capable of binding to cell surface proteoglycans can display enhanced efficiencies of gene transfer. Alternatively, compounds such as fucoidan or heparin that compete with proteoglycan binding of CLDC can be administered to patients to modulate the timing or efficiency of gene delivery by lipid/polynucleotide complexes.

The examples below are provided to illustrate the subject invention. Given the level of skill in the art, one can be expected to modify any of the above or following disclosure to produce insubstantial differences from the specifically described features of the present invention. As such, the following examples are provided by way of illustration and are not included for the purpose of limiting the invention.

### EXAMPLES

### Example

### Construction of Lipid/Polynucleotide Complexes

a. Reagents

Reagent grade DOTIM was obtained from Dr. Tim Heath, and cholesterol from Calbiochem. Particularly where in vivo use is contemplated, all reagents will be of the highest purity available, and preferably of pharmaceutical grade or better.

b. Vector Construction

Plasmid p4331 was constructed by ligating the HindIII+AccI DNA fragment of p630 containing the EBNA-1 cDNA (Middleton and Sugden, 1992, J Virol 66:489-495.), into the HindIII-AccI sites of HCMV-CAT, p4119 (Liu et al., 1995, supra.). Plasmid 4395 was constructed by isolating the HindIII+AccI DNA fragment of p630, and inserting it by blunt end ligation into the EcoRV+BamHI site of VR1255, a gift from Drs. P. Felgner and R. Zaugg (Hartikka et al, 1996, Hum. Gene Ther. 7:1205-1217). Plasmid 4329 was constructed by partially digesting p985 (Middleton et al., 1992, supra.) with BamHI, and then with KpnI, and then ligating the approximately 3 kb DNA fragment containing family of repeat sequences upstream of the TK promoter linked to the luciferase cDNA into the BamHI-KpnI site of p4119 (Liu et al., 1997, supra.). Plasmid p4379 was constructed by digesting p985 (Middleton et al., 1992, supra.) with BamHI, and then isolating the approximately 0.9 kb DNA fragment containing FR, and ligating it into the BamHI site (3′ to the luciferase cDNA) of VR1225 (Harttika, 1996).

Plasmid p4402, CMV-hG-CSF-FR was constructed by first inserting the 0.9 kbp BamHI DNA fragment from p985 containing FR into the site of VR1223 (Hartikka et al., 1996, Hum. Gene Ther. 7:1205-1217.), from Vical, and then replacing the approximately 1.7 kbp PstI-Xba I luciferase cDNA from VR1223 with a HindIII-SalI fragment containing the 650 bp human G-CSF cDNA from p4195 (Liu et al., 1994, supra.) by blunt end ligation. Construction of p4195, an HCMV IE1-human G-CSF expression plasmid (Liu et al., 1995, supra.), oriP-BamHI-Luc (p1033), oriP-minus (p1381), competitor DNA (p1380) (Kirchmaier and Sugden, 1997, J. Virol. 71:1766-1775) and HCMV-luc-AAV-ITR (Philip et al., 1994, Mol Cell Biol. 14(4):2411-8), were reported previously. Plasmids were purified using alkaline lysis and ammonium acetate precipitation as described previously (Liu et al., 1995, supra.).

c. Protocol for Formulating Lipid/Polynucleotide Complexes

The cationic lipid 1-[2-(9(Z)-Octadecenoyloxy)ethyl]-2 -(8(Z)-heptadecenyl)-3-(2-hydroxyethyl)-imidazolinium chloride (DOTIM) was synthesized as previously described (Solodin, 1995), and cholesterol was purchased from Sigma (St. Louis, Mo.). DOTIM:cholesterol multilamellar vesicles were prepared in a 1:1 molar ratio essentially as previously described (Liu et al., 1997, supra.).

d. In Vitro Gene Delivery

Gene delivery complexes formed using the described vectors and methods can be added primary target cell cultures, secondary cell cultures, embryonic stem cell cultures, cell lines, transformed cell lines, tumor cells lines, and the like. Typically, the gene delivery complexes are added at a vector polynucleotide:target cell ratio of about 2 μg polynucleotide:about 100,000 to about 500,000 cells. The gene delivery complexes are typically added to the cell culture medium and can be incubated from about 30 minutes to indefinitely.

e. In vivo Gene Delivery

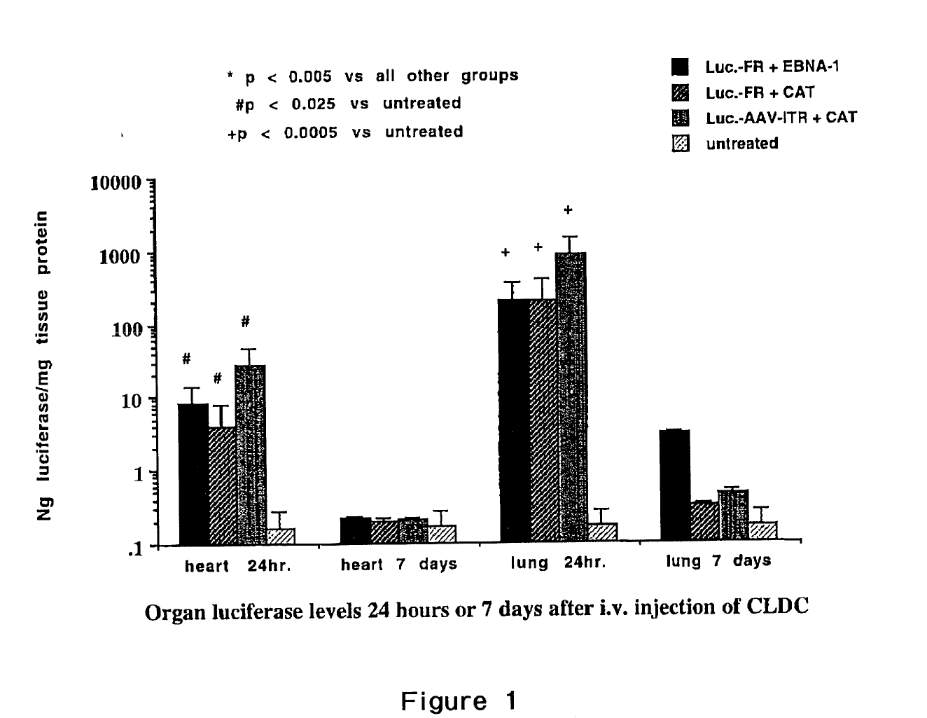

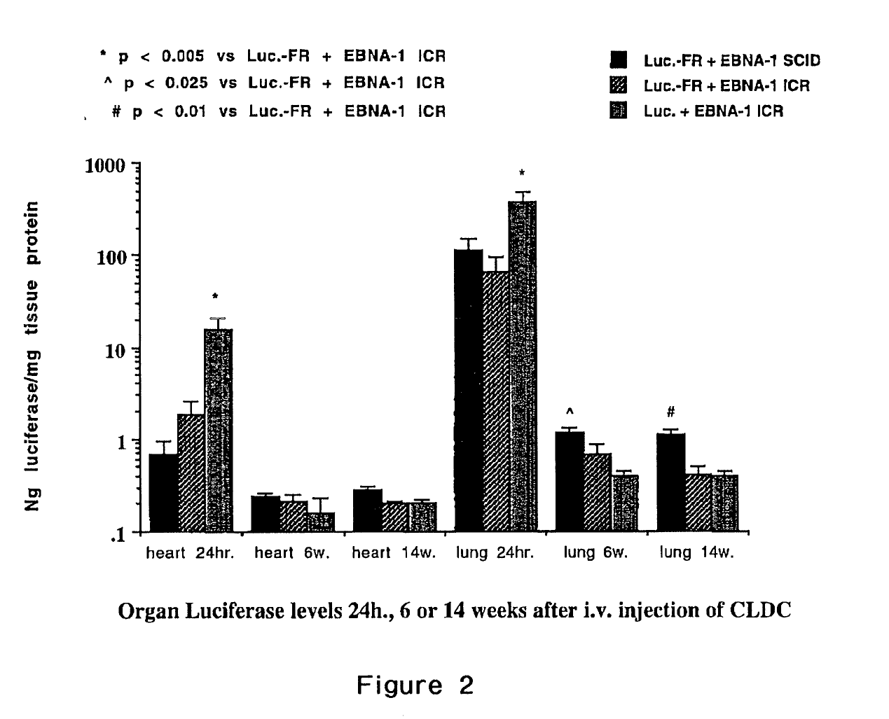

Individual mice in groups of four were injected intravenously with CLDC prepared from 30 μg of each plasmid for a total of 60 μg of DNA. Prior to injection, the two DNAs were mixed together, and then complexed to cationic liposomes in 5% w/v glucose at a plasmid DNA:cationic liposome ratio of 1 μg DNA:16 nanomole total lipid, as described previously (Liu et al., 1995, supra.). CLDC were injected by tail vein in a total volume of 200 μl per mouse. From 24 hours to 15 weeks following i.v. injection of CLDC, groups of mice were sacrificed by exposure to CO₂, bled via cardiac puncture, tissues dissected, and luciferase activity (Liu et al., 1997, supra.) or hG-CSF protein levels (Liu et al., 1995, supra.) were performed as previously described. Total white blood cell counts were determined with a hemacytometer using EDTA anticoagulated blood diluted in a Unopette white cell test system (Becton Dickinson, Franklin Lakes, N.J.). Differential counts were performed by an individual blinded to the experimental design using blood smears stained with Diff-Quik (Scientific Products, McGaw Park, Ill.). The results of these studies are shown in FIGS. 1-3.

The duration of gene expression was measured after i.v. co-injection of a CLDC containing an expression plasmid containing EBV-FR DNA sequences plus either the luciferase or hG-CSF cDNAs. This plasmid did not contain an EBV region of dyad symmetry (DS) and therefore lacked an intact oriP and could not replicate in cells, i.e., was replication defective. This plasmid was co-delivered with an expression plasmid that transiently expressed the EBNA-1 gene and lacked EBV-FR sequences. Despite their inability to replicate in mice, and despite the only transient expression of the EBNA-1 gene, this vector system produced significant levels of luciferase activity for at least 14 weeks, as well as therapeutic serum levels of hG-CSF protein for at least two months following a single, CLDC-based i.v. co-injection. Furthermore, despite producing long term expression of gene products which are immunogenic in mice, both the luciferase and the hG-CSF genes could be re-expressed in immunocompetent mice, following repeat i.v. co-injection of CLDC containing these EBV-based vectors.

Previously, it had been reported that the insertion of AAV-ITR sequences into plasmid vectors significantly prolonged gene expression in cultured cells (Philip et al., 1994, supra.). However, co-injection of the HCMV-luc-AAV-ITR plasmid with an HCMV plasmid expressing the AAV-rep gene did not extend luciferase expression when compared to HCMV-luc-AAV-ITR plus HCMV-CAT. These results indicate that the presence of EBNA-1 prolongs the expression of luciferase in mouse lungs from a plasmid containing FR, when compared to either the absence of EBNA-1, or to an AAV-ITR-based vector. In all experiments, mice that did not receive the combination of an FR-containing plasmid together with an EBNA-1 expression plasmid did not display tissue luciferase activity significantly above background at or beyond 14 days post injection.

The level of luciferase activity produced in either immunocompetent ICR mice or SCID mice was compared 24 hours, six weeks and 14 weeks following i.v. injection of CLDC containing p4329, HCMV-luciferase-FR and p4331, HCMV-EBNA-1. To test the effects of EBNA-1 in the absence of FR, luciferase activity was measured in ICR mice receiving HCMV-EBNA-1 and p4241, HCMV-luc which lacked FR. Although the amount of luciferase in lung and heart tissue was similar in immunocompetent and SCID mice sacrificed 24 hours after injection, luciferase activity in SCID mice was significantly higher than in immunocompetent ICR mice sacrificed six weeks after i.v. co-injection (p<0.025). The amount of luciferase in lung tissue remained significantly higher in SCID mice than in either ICR mice treated with the same EBV-based plasmids (p<0.01) or in untreated control mice (p<0.005), 14 weeks after a single i.v. injection. These results indicate that EBNA-1 does not prolong the expression of HCMV-luc in the absence of FR, consistent with the interpretation that EBNA-1 is functioning via its ability to retain FR-containing plasmids intracellularly (Middleton et al, 1994, supra.), rather than by affecting expression from the HCMV promoter. However, peak levels of luciferase produced at 24 hours were similar in mice receiving HCMV-luc plasmids with or without FR which suggests that FR does not function as a transcriptional enhancer in the presence of EBNA-1 in mice, as had been predicted by in vitro studies using cultured cells.

The fact that luciferase levels in both SCID and ICR mice receiving HCMV-luc-FR plus HCMV-EBNA-1 were similar at 24 hours, but were significantly higher in SCID mice at 6 and 14 weeks post injection indicated that an immune response directed against the luciferase gene product can limit the duration of luciferase gene expression produced in immunocompetent ICR mice. Host immune responses directed against luciferase as well as other reporter gene products have been reported previously (Mittal et al., 1993, Virus Res., 28:67, Michou, 1997, Gene Ther., 4:473-482).

To assess whether such an immune response would significantly hinder subsequent treatments, a second i.v. injection of p4379, HCMV-luciferase-FR, together with p4331, HCMV-EBNA-1 was administered to the same ICR mice that had been transfected with the same CLDC 31 days earlier. I.v. re-injection of these EBV-based plasmids produced efficient re-expression of luciferase (FIGS. 3a and 3b). In fact, the levels of luciferase observed one day after the second CLDC injection on day 31 were more than 100 fold higher than those observed in mice sacrificed 31 days after a single injection of CLDC, and did not differ significantly from peak luciferase levels produced in mice receiving a single i.v. injection of CLDC containing the EBV-based plasmids 24 hours earlier (3a). These results indicated that protein expression from EBV-based, long expressing plasmids can be re-established by re-injection of the same CLDC. This result is consonant with previous i.v. re-injection studies using CLDC mediated delivery of non-EBV-based plasmids (liu et al., 1995, supra.). For example, multiple reinjection of p4395, the HCMV-EBNA-1 vector does not decrease the efficiency of CLDC-based, IV gene delivery, indicating that the presently described EBV-based system can continuously re-express delivered genes in fully immunocompetent hosts for very prolonged periods. In these experiments, plasmids p4379 (HCMV-luciferase-FR), p4395, HCMV-EBNA-1 and p4119, and/or HCMV-CAT were formulated in to DOTIM:cholesterol MLV in 1:1 molar ratio (DNA:lipid ratio=1:16 (μg plasmid DNA to nanomoles total lipid), and forty μg plasmid DNA in 200 μl of 5% dextrose in water (D5W) were injected by tail vein per animal (25 gram ICR female mice Simonsen Labs, Gilroy, Calif.). One group of mice received two injections of CLDC containing 20 μg of CMV-CAT plus 20 μg of CMV-EBNA-1 at 3 week intervals, then received an injection of 20 μg of CMV-luciferase-FR plus 20 μg of CMV-EBNA-13 weeks later, and were sacrificed 3 weeks after the last injection. CMV-CAT was co-injected with CMV-EBNA-1 for the first two injections in order to prevent the induction of an immune response against the indicator molecule (luciferase). A second group of mice received one injection of CLDC containing 20 μg of CMV-EBNA-1 plus 20 μg of CMV-LUCIFERASE-fr and were sacrificed 3 weeks later. A third group of mice received a single dose of CLDC containing 20 μg of CMV-CAT plus 20 μg of CMV-luciferase-FR and were sacrificed 3 weeks later. A fourth group were left untreated (controls). All mice were sacrificed in a CO₂ chamber, and lungs, heart, spleen, and liver were collected and assayed for luciferase activity. Relative light units were converted to luciferase activity, and an unpaired, two side Student's T test applied for statistical analysis of potential differences between groups as described previously (Liu et al., NatBioT, 1997).

The levels of luciferase activity in the groups of mice that received either a single dose of CMV-EBNA-1, or a total of 3 doses of CMV-EBNA-1 were comparable, and did not differ significantly (p<.4) (see FIG. 3b). Luciferase activity was significantly higher in these two groups than in mice receiving CMV-luciferase-FR co-injected with CMV-CAT, indicating that the CMV-EBNA-1 plasmid was still able to mediate long-term expression of a co-injected CMV-luciferase-FR plasmid even in mice that had received 2 prior injections of CMV-EBNA-1 over the previous 6 weeks. Thus, repeated injection of the CMV-EBNA-1 plasmid in fully immunocompetent mice did not reduce long-term expression of a CMV-luciferase-FR plasmid subsequently co-injected with CMV-EBNA-1. This indicated that there does not appear to be an immune response against EBNA-1 in mice repeatedly injected with CMV-EBNA-1 and suggests that the presently described EBV-based system can produce significant levels of gene transfer and expression for the lifetime of the host.

The data presented in FIGS. 3a and 3b indicate that, unlike viral or other inherently antigenic/immunogenic gene delivery vehicles, the presently described CLDC can be repeatedly used to effect gene delivery with less concern about the host immune response to the gene delivery vehicle unduly affecting the expression of the delivered genes.

Previous studies had shown that oriP-containing plasmids do not replicate in the presence of EBNA-1 in rodent cell lines. To determine whether EBV-based plasmids containing an intact oriP could replicate in primary murine lung tissue, mice were injected i.v. with 20 mg each of oriP-BamHI C-Luc, oriP-minus and either p4331, HCMV-EBNA-1 or p4241 (HCMV-luc). After 14 days, the mice were sacrificed and low molecular weight DNA was isolated from lung tissue. The data in Table 1 indicate that although both oriP-BamHI C-Luc and oriP-minus DNAs were present in mice lungs 14 days after i.v. injection, neither plasmid was detectably replicated in either the presence or the absence of EBNA-1. In contrast, oriP-BamHI C-Luc efficiently replicated in the presence of EBNA-1 in human PPC-1 cells (Table 1). However, oriP-BamHI C-Luc was not detectably replicated in the absence of EBNA-1 at 96 hours post-transfection in PPC-1 cells (Table 1). These results indicate that EBV-based plasmids containing an intact oriP do not detectably replicate in primary murine tissue in the presence or absence of EBNA-1. Previously, a BKV-based expression plasmid has been shown to replicate in mouse lungs, two weeks after i.v., CLDC-based injection in mice (Thierry, 1995, Proc. Natl. Acad. Sci., USA, 92:9742-9746), demonstrating that such replication is possible if appropriate sequences are present.

Since the presence of the EBNA-1 protein might facilitate nuclear entry/delivery of the oriP-FR containing plasmid shortly after it enters the cell, i.v. co-injection of HCMV-luciferase plus oriP-FR together with HCMV-EBNA-1 was compared to pre-injecting HCMV-EBNA-1 6, 24 or 48 hours prior to injecting HCMV-luciferase plus oriP-FR. Measurements taken two weeks after i.v. injection of CLDC indicated that only co-injection of the 2 plasmids produced levels of tissue luciferase activity significantly above background. This appeared to be due to a temporary inability to efficiently retransfect mice by i.v. reinjection of CLDC in the first several days following the initial i.v. injection.

Table 1. oriP-based vectors are not detectably replicated in the presence of EBNA-1 in primary murine tissue.

TABLE 1

| | DpnI-digested^{a} | Nonddigested^{a} | |
|---|---|---|---|
| Cells/ | oriP-BamHI C-Luc/ | oriP-BamHI C-luc/ | oriP-minus/ |
| Effector | 1 × 10⁵ cells | 1 × 10 cells | 1 × 10⁵ cells |
| Lung/ | <8600 ± 6400^{b} | 2.2 × 10⁴ ± | 3.7 × 10⁴ ± |
| EBNA-1 | | 0.9 × 10⁴ | 0.97 × 10⁴ |
| Lung/pLuc | <16000 ± 6400^{b} | 2.6 × 10⁴ ± | 3.7 × 10⁴ ± |
| | | 3.8 × 10⁴ | 1.3 × 10⁴ |
| PPC1/ | 2.1 × 10⁵ ± | —^{c} | — |
| EBNA-1 | 0.34 × 10⁵ | | |
| PPC-1/pLuc | <4930 | — | — |

^{a}Data represents molecules of plasmid DNA per 1 × 10⁵ cells. Data has not been corrected for the transfection efficiency of lung tissue or of the PPC1 cell line.
^{b}Less than the lowest amount of competitor DNA detected.
^{c}Not tested.

f. Circulating levels of human G-CSF protein following i.v. injection of CLDC containing the hG-CSF gene

The EBV-based two plasmid system (containing the FR but lacking the region of dyad symmetry) was also used to demonstrate both the prolonged expression of the biologically relevant hG-CSF gene, and the re-expression of hG-CSF following a second injection in immunocompetent mice. The levels of hG-CSF in mouse serum were measured by ELISA following i.v. injection of CLDC containing either p4402 or p4195, HCMV-hG-CSF with or without FR, respectively, together with p4395, an HCMV-EBNA-1 plasmid. As shown in Table 2, ICR mice injected with the hG-CSF expression plasmid plus FR expressed 4,861±2,606, 636±45, 457±86 and 187±74 pg/ml of hG-CSF in mouse serum at days one, 14, 31 and 62 after injection respectively. In contrast, mice injected with a hG-CSF vector lacking FR plus HCMV-1-EBNA expressed 5,274±3,333 pg/ml of hG-CSF protein in their serum at day one, but hG-CSF levels were not detectable (below 25 pg/ml) at days three and seven following injection (Table 2).

TABLE 2
Significant levels of human G-CSF protein are maintained in the serum
of ICR mice for prolonged periods following hG-CSF gene delivery
via iv injection of CLDC containing EBV-based plasmid vectors.

| | Days post | hG-SCF in serum^{b} |
|---|---|---|
| Vector^{a} | injection^{a} | (pg/ml) |
| hG-CSF-FR | 1 | 4,861 ± 2,606^{d} |
| ″ | 14 | 636 ± 45^{d,f} |
| ″ | 31 | 457 ± 86^{d} |
| ″ | 31, +1c | 1,192 ± 264^{d,e} |
| ″ | 62 | 187 ± 74^{d,g} |
| ″ | 62, +1c | 1,151 ± 250^{d,e} |
| hG-CSF | 1 | 5,274 ± 3,333^{d} |
| ″ | 3 | <20 |
| ″ | 7 | <20 |
| No DNA (uninjected) | 0 | <20 |

^{a}CLDC containing 30 μg of indicated vector plus 30 μg of HCMV-EBNA-1 were injected iv into groups of 4 ICR mice on day 0. Mice were sacrificed and bled at days indicated.
^{b}Serum levels of hG-CSF were measured by ELISA (Liu, JBC). Data represents mean ± S.E.M. for four mice per vector and time point.
^{c}Groups of 4 mice were re-injected as described in “a” at either 31 or 62 days following their first dose of the hG-CSF vector, and sacrificed 24 hours after the second injection.
^{d}p <0.05 when compared to control mice by a two-sided Student's t test.
^{e}p <0.05 when compared to non-redosed mice injected simultaneously.
^{f}the respective percent increases for absolute neutrophil counts (ANC) or band counts versus untreated controls were 477 ± 55 and 2.0 ± 1.1, p <0.005.
^{g}the respective percent increases for absolute neutrophil counts (ANC) or band counts versus untreated controls were 457 ± 86 and 3.0 ± 2.0, p <0.005.

Previously, sustained serum levels of hG-CSF above 100 pg/ml had been shown to significantly increase neutrophil counts in rodents (21) (Koeberl, 1997). Therefore, both the percentage of neutrophils and the absolute numbers of neutrophil per ml of whole blood were measured in mice that received a single i.v. injection of CLDC containing either CMV-hG-CSF-FR or CMV-luc-FR plus CMV-EBNA-1 eight weeks earlier, as well as in untreated mice. Mice receiving CMV-luc-FR plus CMV-EBNA-1 or no treatment showed 9.4±1.3% or 8.9±1.3% neutrophils with a complete absence of band (immature neutrophil) forms and absolute neutrophil counts of 551±90 or 673±58 per mm³ of blood, respectively, whereas mice receiving CMV-hG-CSF-FR plus CMV-EBNA-1 showed 24.0±2.5% neutrophils with 1% band forms and absolute neutrophil counts of 2,805±488 mm³ of blood (p<0.005 versus either CMV-luc-FR treated or untreated mice for both the percentage of and the absolute number of neutrophils, Table 2). Mice sacrificed 2 weeks after a single i.v. injection of CMV-hG-CSF-FR plus CMV-EBNA-1 or no treatment showed similar elevations of both the percentage and absolute number of neutrophils versus either luciferase injected or uninjected mice (p<0.005 for both) indicating that this effect was sustained over the 8 week period. Taken together, these results indicate that biologically significant levels of hG-CSF can be expressed for prolonged periods from EBV-based vectors in mice. Furthermore, the level of hG-CSF was significantly increased (p<0.05) 24 hours after a second injection of HCMV-hG-CSF-FR together with HCMV-EBNA-1 in ICR mice that had been expressing hG-CSF at therapeutically relevant levels for the preceding two months (Table 2).

The above data indicates that a non-replicating EBV-based two plasmid system can both increase the cellular retention of FR-containing plasmids (Middleton et al., 1994, supra.), and mediate their binding to the nuclear matrix (Jankelvich, 1992), with CLDC-based i.v. gene delivery that preferentially targets gene expression to vascular endothelial cells (Liu et al., 1997, supra.), a cell type that is largely non-dividing in normal adults (Denekamp, 1982, Bicknell, 1992). The CLDC-based delivery of these EBV plasmids significantly extends the duration of gene expression and allows for the re-expression of genes coding for potentially immunogenic proteins (Bonham, 1996) in immunocompetent mice. This approach utilizes EBNA-1 as the viral DNA binding protein. Although mice transgenic for EBNA-1 have been reported to develop B cell tumors, (Wilson, 1996), EBNA-1 itself is insufficient in context of the EBV virus to immortalize primary B lymphocytes in vitro, and additionally requires the presence of the latent viral proteins EBNA2 (Hammerschmidt, 1989, Cohen, 1989, Marchini, 1992), EBNA3A (Tomkinson, 1993), EBNA3C. (Tomkinson, 1993) and LMP-1 (Kaye, 1993).

Unlike BKV- or SV40-based, replicating vectors, that utilize ongoing expression of a large T antigen (Thierry, 1995, Proc. Natl. Acad. Sci., USA, 92:9742-9746, Cooper, 1997), the EBNA-1 gene was expressed from a plasmid that lacked FR, and thus only transiently expressed EBNA-1. Despite this approach, which was designed to minimize the transforming potential of EBNA-1, EBNA-1 was able to mediate the durable expression of genes encoded by co-injected FR-containing plasmids. Thus, this EBV-based two plasmid system is the only available long-expressing (more than three days) plasmid vector system that should be satisfactory for human gene therapy.

Another consideration is that in vitro studies are typically conducted using rapidly dividing transformed cells, whereas in vivo applications typically involve cells with much lower rates of cell division (i.e., are effectively nonreplicating). Such considerations can also partially explain why transient expression of a CRA in vivo affords long term expression whereas similar results are not seen in vitro.

This can also be partially explained by its ability to limit EBNA-1-specific cytotoxic T lymphocyte (CTL) responses, mediated by Gly-Ala repeats within EBNA-1 that generate a cis-acting inhibitory signal which interferes with antigen processing and MHC class I-restricted presentation (Levitskaya, 1995, Khanna, 1992, 1995, Murray, 1992). The ability to limit generation of EBNA-1 specific CTL can also contribute to the present systems demonstrated ability to re-transfect immunocompetent mice with either luciferase or hG-CSF after repeat injections of these genes, together with an EBNA-1 expression plasmid (Table 2).

The use of EBV-based plasmids can prove particularly relevant for treatment of inherited genetic diseases such as cystic fibrosis and the hemophilias; diseases which require that the gene transfer vector must express the transferred gene at therapeutic levels for prolonged periods following a single administration, and then efficiently support prolonged expression of that gene following subsequent administrations at regular intervals throughout the lifetime of the patient (Knowles-1995, Sorscher, 1994, Caplen, 1995, Hyde, 1993, Alton, 1993. Zabner, 1993, Snyder, 1997., Kay, 1993). The use of EBV-based plasmids containing FR but lacking an intact oriP can permit targeting of durable gene expression to non-replicating cells in vivo. Furthermore, it is very likely that the prolonged gene expression observed in injected mice (in which EBV vectors do not replicate) can be further amplified in primates, in which the fraction of replicating cells that take up EBV-based plasmids containing an intact oriP will propagate the plasmids.

The coinjection studies also showed that HCMV-luciferase expression plasmids in which the EBV FR DNA sequences were inserted between the heterologous intron and the luciferase cDNA, produced peak levels of luciferase activity 5 to 10 fold lower than the vectors lacking EBV DNA sequence. EBV DNA sequences placed in this position presumably interfere with gene expression; however, this effect is apparently vector specific. Subsequent studies revealed that placement of the EBV sequences downstream from the coding sequence yielded a vector that produced peak levels of luciferase gene expression comparable to that of the parent vector lacking EBV sequences, and significantly higher than vectors containing EBV sequences 3′ of the intron. In fact, a single i.v., CLDC-based co-injection using this more efficient HCMV-luciferase plus FR vector produced significantly increased levels of luciferase activity in both the lungs and heart of immunocompetent ICR mice for at least 12 weeks.

### Example

### Formulation of CLDCs in Different Diluents

As shown by the experiment detailed below, changing the diluents in which DNA and cationic liposomes are complexed can significantly increase the efficiency of CLDC-based, IV gene delivery.

Plasmid: p4241 (HCMV-luciferase).

Liposomes: DOTIM:chol MLV in 1:1 molar ratio.

DNA:Liposome Ratio: liposome:plasmid=1:16 (μg plasmid DNA to nanomoles total lipid).

Preparation of CLDC: CLDC were formulated in four different diluents: formulas A, B, C, and D. Formula A was prepared as follows: 40 micrograms plasmid DNA and 640 nanomoles rhodamine-labeled DOTIM:chol MLV were each diluted in 100 μl D5W, then mixed together as described previously (Liu et al. JBC, 1995). For Formula B, plasmid DNA was diluted in a solution containing dextran 40 and Ringer's lactate at a ration of 9:1, and the cationic liposomes were diluted in pure Ringer's lactate. Formula C is similar to Formula B except plasmid DNA is diluted in fetal bovine serum (Gibco) instead of in a 9:1 mixture of dextran 40 and Ringer's lactate. For Formula D, the plasmid DNA was diluted in 70 μl of fetal bovine serum and the liposomes in 70 μl of D5W. After mixing the DNA and liposomes together, 60 μl of Ringer's lactate solution was added to the CLDC and pipetted gently twice to mix.

DNA dose: 40 μg plasmid DNA in 200 μl of Formula A, B, C, or D was injected by tail vein per mouse.

Animals: ICR mice:female, 25 grams.

Quantitation of luciferase Twenty-four hours after injection of CLDC, mice were sacrificed in a CO₂ chamber, and lungs, heart, spleen and liver were collected and assayed for luciferase activity, relative light units converted to luciferase activity, and an unpaired, two side Student's t test applied for statistical analysis as described previously (Liu et al., 1997).

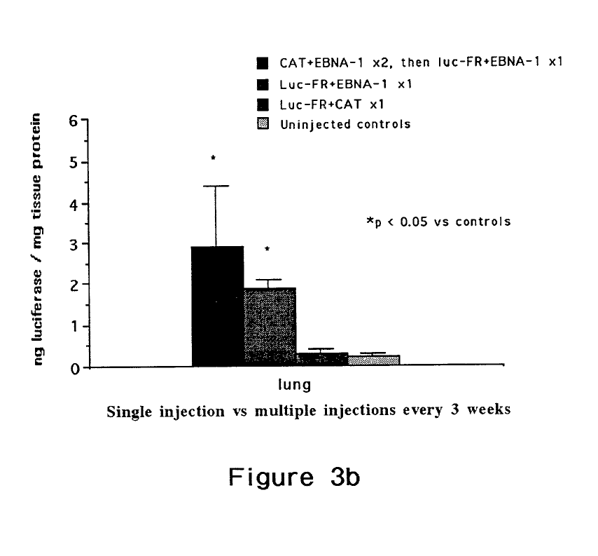

Results: Preparation of CLDC in either Formulas B, C or D above significantly increased luciferase when compared to luciferase activity produced in mice in which CLDC injected IV were prepared in D5W (FIG. 4). Thus, selectively changing the materials in which DNA and cationic liposomes are diluted can significantly increase the level of gene expression produced in animals subsequently injected in with CLDC. The level of enhancement of gene expression produced by these diluents was significantly greater in Swiss Webster mice (low expressors) than in ICR mice (high expressors) suggesting that such manipulations can be particularly useful in individuals who exhibit low levels of gene expression following intravenous injection of CLDC in D5W or other standard diluents.

### Example

### Strain Variability in In Vivo Gene Delivery

This example illustrates that the strain of mice in which CLDC are injected intravenously plays an important role in determining the efficiency of gene delivery and expression.

Plasmid: p4241 (HCMV-luciferase).

Liposomes: DOTIM:chol MLV in 1:1 molar ratio.

DNA:Liposome Ratio: liposome:plasmid=l:16 (μg plasmid DNA to nanomoles total lipid).

DNA dose: 40 μg plasmid DNA in 200 μl of 5% dextrose in water (D5W) was injected by tail vein per mouse.

Animals: Three different mouse strains were compared in this experiment for luciferase gene expression, rhodamine-labeled liposome distribution and luciferase DNA recovery (by Southern analysis) from tissues after CLDC-based IV gene delivery. Six week old female ICR, FVB and Swiss Webster mice were purchased from Simonsen Labs, Gilroy, Calif.

Quantitation of luciferase. Twenty-four hours after injection of CLDC, mice 20 were sacrificed in a CO₂ chamber, and brain, lungs, heart, spleen and liver were collected. A portion of liver and lung tissues from the same mouse were quick frozen in dry ice and reserved for Southern analysis and fluorescence assay and the remaining tissues were assayed for luciferase activity, relative light units converted to luciferase activity, and an unpaired, two side Student's t test applied for statistical analysis as described previously (Liu et al., 1997). Lipid was extracted from the tissue, levels of rhodamine fluorescence determined and Southern analysis performed as previously described in (Liu et al., 1997).

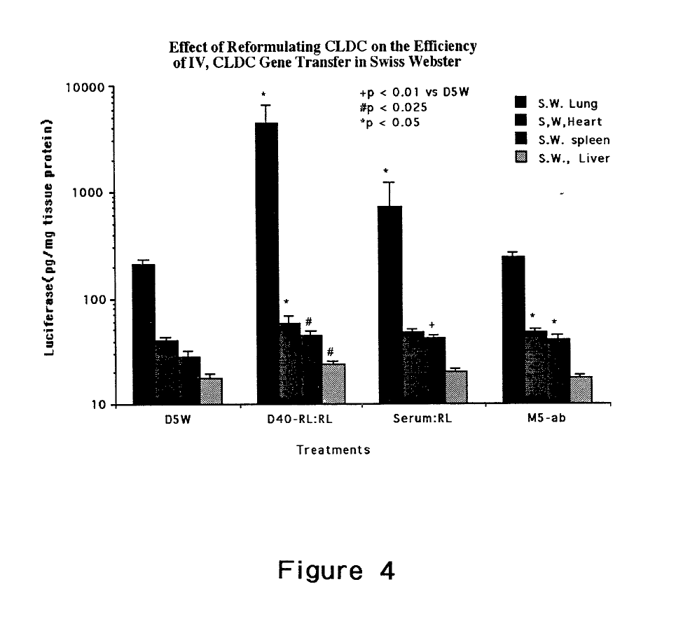

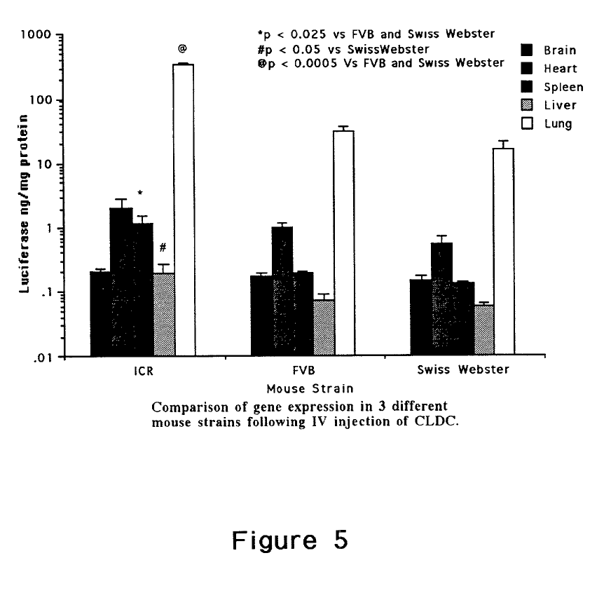

Results. I.V. injection of identical CLDC containing the luciferase gene into 3 different strains of mice produced very different levels of luciferase gene expression. The level of luciferase gene expression produced in ICR mice was significantly higher than that produced in either FVB or Swiss Webster strains of mice (FIG. 5). Thus, the level of gene expression produced by IV injection of CLDC is significantly higher in some strains of mice than in other strains, indicating that there are high expressor and low expressor variants for in vivo gene transfer. As demonstrated in FIG. 6, low expressor strains can be converted to higher expressor variants by changing the diluents for the plasmid DNA and cationic liposome components. Similar strategies can be used for dealing with low expressor human patients.

### Example

### Host Pre-treatment

This example demonstrates that pretreatment with either dexamethasone or 4-APP or other selected agents significantly increases the efficiency of CLDC-based, IV gene delivery. Additionally, efficiency of delivery of naked DNA can also be enhanced with host pretreatment.

Plasmid: p4241 (HCMV-luciferase, see Liu et al., 1997).

Liposomes: DOTIM:chol MLV in 1:1 molar ratio.

DNA:Liposome Ratio: liposome:plasmid 1:16 (μg plasmid DNA to nanomoles total lipid).

Pretreatment: Individual mice in groups of 5 received either 200 μl of D5W only by IV injection, 1 mg dexamethasone (Sigma) in 200 μl D5W by IV tail vein injection, 250 μg Ticlopidine (Sigma) dissolved in 200 μl D5W by IV injection, 175 μg ammonium chloride (Fisher) dissolved in 1000 μl D5W by IP injection, respectively, four hours before IV injection of CLDC. For 4-APP pretreatment, a group of 5 mice were IP injected with 1.5 mg of 4-Aminopyrazolo(3,4d)-pyrimidine (Sigma, A2630) dissolved in 1 ml 0.01 M sodium phosphate, pH 2.5, daily for three days before receiving an IV injection of p4241-containing CLDC as described above. Another group of 5 mice received 1 ml 0.01 M Sodium Phosphate, pH 2.5 solution by IP injection daily for three days before receiving an injection of IV CLDC as above, thus serving as control for 4-APP pretreatment group.

DNA dose: 40 μg plasmid DNA in 200 μl of 5% dextrose in water (D5@) was injected by tail vein per mouse.

Animals: ICR mice:female, 25 grams (Simonsen Labs, Gilroy, Calif.).

Quantitation of luciferase: Twenty-four hours after injection of CLDC, mice were sacrificed in a CO₂ chamber, and lungs, heart, spleen and liver were collected and assayed for luciferase activity. Relative light units were converted to luciferase activity, and an unpaired, two side Student's t test applied for statistical analysis of potential differences between groups as described previously (Liu et al., 1997).

Results: Preinjection of either dexamethasone intravenously or 4-APP by intraperitoneal injection significantly increased luciferase activity in groups of mice injected iv with CLDC when compared to luciferase activity produced in groups of mice pretreated with either buffer only, or with a variety of other compounds (see FIG. 6). Thus, preinjection of either dexamethasone or 4-APP can significantly increase the level of gene expression produced in animals subsequently injected iv with CLDC.

In another experiment, the effect of pretreatment on subsequent expression of the same plasmids delivered as naked DNA was examined. Pretreated animals were injected with 6.25 mg of mannitol (Sigma, MO) in 200 microliters of water 2 hours or 24 hours prior to iv administration of the p4241 luciferase vector as naked DNA in water, 50% glycerol or D5W. Control animals received water alone as a pretreatment. Subsequent in vivo expression of luciferase from p4241 was significantly higher in animals treated with mannitol 2 hours prior to administration of p4241 as compared to controls, particularly those animals receiving the expression plasmid in D5W. Similar increases in expression from mannitol treated animals were observed when the vector was administered as a CLDC as described above. Some increase in luciferase expression was also seen in animals receiving mannitol 24 hours prior to p4241.

Discussion: This experiment demonstrates that host pretreatment can affect subsequent levels of expression from delivered gene therapy vectors. Comparison of the differences between treated and untreated hosts will elucidate those factors and pathways rate limiting to transgene expression. Such pathways and factors can be manipulated to increase efficiency. For example, differential gene expression can be analyzed using any of a number of techniques including but not limited to SAGE (Veculescu et al., 1995, Science 270:484) and genome-wide gene expression (Eisen et al., 1998, Proc. Natl. Acad. Sci. U.S.A. 95:14863).

### Example

### Identification of Cell Surface Receptor for Cationic Lipid/Polynucleotide Complexes

Proteoglycans perform a wide variety of functions ranging from formation of extracellular matrix to cell-cell contact and communication. Proteoglycans also function in the binding and entry of many viruses into cells, including herpes simplex virus, murine cytomegalovirus, and HIV-1. Proteoglycans can also act as reservoirs for growth factors and in some cases can regulate growth factor function, e.g. bFGF. Further illustrating the diversity of functions encompassed by these proteins, proteoglycans are also involved in the regulation of lipid metabolism and in the binding of monocytes to subendothelial matrix.

Proteoglycans have been shown to mediate gene transfer into cultured cells in vitro by methods relying on poly-lysine or cationic liposomes (Mislick and Baldeschwieler, 1996). This observation suggests that cell surface proteoglycans can play a role in the uptake of CLDC in vivo. However, it has also been shown that soluble heparin sulfate can release DNA from CLDC in vitro, suggesting that glycosaminoglycan (GAG)-bearing proteoglycans present in serum or interstitial fluids can block CLDC-based transfection by releasing DNA from CLDC and preventing internalization of the DNA (Xu and Szoka, 1996; Zelphati and Szoka, 1996). Furthermore, results in vitro systems are notoriously unreliable in predicting the results obtained when by identical gene delivery approaches are used in vivo (Liu et al., 1997). The role of proteoglycans in mediating CLDC-based gene delivery in vivo, is therefore, unknown.

As discussed below, proteoglycans play a significant role in CLDC-mediated delivery and expression of heterologous genes both in vitro and in vivo. In particular, the proteoglycan syndecan-1 has been implicated as a mediator of gene transfer in vitro. Heparinase I pretreatment of animals demonstrates the specific importance of heparin sulfate proteoglycans in CLDC-based, intravenous transfection in mice in vivo. Pretreatment of animals with either polysaccharides (fucoidan or heparin) or heparinase I in vivo severely limits CLDC mediated gene transfer and expression by limiting cellular uptake of CLDC. These data indicate that CLDC bind to cell surface proteoglycans prior to transferring the complexed DNA into the cell.

a. MATERIALS AND METHODS

Plasmids. The construction of p4241 has been described (Liu et al., 1997). Plasmids were purified as previously described (Liu et al., 1997).

Preparation of cationic liposomes and CLDC. DOTIM:DOPE SUV and DOTIM:Chol MLV were prepared as previously described (Liu et al., 1997). CLDC were prepared as described (Liu et al., 1995).

In vitro transfections. For CLDC transfections in vitro, 1-2×10⁵ cells were plated per well in 12- or 24-well plates. Cell types used were hamster CHO cells, mouse B16 melanoma cells, the human prostate cancer lines PPC-1 and DU-145, the human breast cancer line MDA-435, Raji cells, and Raji cells stably transfected with syndecan-1. CHO cells were grown in Ham's F-12 with 10% fetal bovine serum (FBS). B16 and PPC-1 cells were grown in RPMI-1640 with 5% and 10% FBS respectively. DU-145 and MDA-435 were grown in MEM Eagle's with Earle's BSS/10% FBS and Liebovitz's L15/10% FBS, respectively. Raji wild type and syndecan-1 stably transfected Raji (S1-Raji) cells were cultured in RPMI-1640/10% FBS, supplemented with 300 μg/ml hygromycin B for S1-Raji. Cells were grown at 37° C. with 5% CO₂, with the exception of MDA-435 which was grown without CO₂. Cells were transfected as previously described (Liu et al, 1997).

Prior to CLDC transfection, cells were treated with fucoidan (50,000 M.W.), dextran (40,000 M.W.), or dextran sulfate (500,000 M.W.) purchased from Sigma (St. Louis, Mo.). Heparin was purchased from SoloPak Laboratories, Inc. (Elk Grove Village, Ill.). All luciferase assays were performed as described (Liu et al., 1997).

B16 cells were electroporated with p4241 as per BioRad (Hercules, Calif.) instructions for the GenePulser II. Calcium phosphate transfections were performed as described in the manufacturer's instructions (Gibco BRL, Grand Island, N.Y.). Adenoviral infection of PPC-1 cells was performed as previously described (Graham and Prevec, 1991).

In vivo transfections. Each mouse received 50 μg p4241 complexed to DOTIM:cholesterol MLV containing 1 mole % rhodamine-PE at a ratio of 1:16 (μg DNA per nmole total lipid). Approximately 25 g ICR female mice (Simonson, Gilroy, Calif.) received 200 μl of CLDC intravenously by tail vein injection. Pretreatments of mice were also by intravenous tail vein injections. Fucoidan, dissolved in phosphate buffered saline (PBS), was pre-injected at a dosage of 500 μg per mouse 0, 1, 2, 10, 24, or 48 hrs prior to CLDC injection. Heparinase I and III (Sigma) were dissolved in 0.15 M sodium chloride at a concentration of 75 units per 100 μl, and 100 μl per mouse were injected 15 minutes prior to injection of CLDC. Control mice were preinjected at appropriate times with either PBS or 0.15 M sodium chloride. As an additional control, heparinase I was boiled for 10 minutes to denature and deactivate the enzyme prior to pre-injection. Mice were harvested 24 hours post-CLDC injection, and pieces of lung, liver, heart, and spleen were placed in 1X lysis buffer (Promega, Madison, Wis.) on ice for luciferase assays (Liu et al., 1997). Samples of liver and lung were frozen on dry ice prior to Bligh Dyer extractions for rhodamine-liposome fluorescence analysis and isolation of DNA for Southern analysis as described (Liu et al., 1997).

Southern analysis. Isolation of total DNA from mouse tissues was performed as described (Liu et al., 1997). Approximately 2×10⁶ B16 cells were cultured in 100 mm dishes for each DNA extraction from cultured cells. Two hundred nanograms of DNA per lane were fractionated on 1% agarose/1X TAE gels overnight. Blotting onto Hybond membrane (Amersham, Arlington Heights, Ill.), prehybridization, hybridization, and washes were performed as described (Sambrook et al., 1989). A 1.2 kb HindIII/EcoRV gel-purified fragment of luciferase from p4241 was radioactively labelled using the Random Primed DNA Labeling Kit (Boehringer Mannheim, Indianapolis, Ind.). Nuclei were isolated from 2×10⁶ B16 cells as described (Sambrook et al., 1989), and lysed in NP40 lysis buffer (10 mM Tris, pH 7.4, 10 mM sodium chloride, 3 mM magnesium chloride, and 0.5% nonidet P-40). Nuclear DNA was then isolated from washed nuclei using the same technique as for total DNA isolation (Sambrook et al., 1989).

DNase protection. One ml of CLDC was made at a DNA to lipid ratio of 1:8, such that the final concentration of DNA was 2 μg/100 μl. CLDC were made with either DOTIM:DOPE SUV or DOTAP:DOPE SUV. One hundred microliters of the resulting complex was treated with 1 μM fucoidan or 1 μM dextran sulfate for 10 minutes or left untreated, and then subjected to DNase I digestion with 10-20 units DNase I (Boehringer Mannheim) for periods of 5, 30 or 90 minutes. Two micrograms of DNA in 100 μl, uncomplexed to liposomes, were treated with 10-20 units DNAse I for 90 minutes. CLDC and naked DNA were made in 5% dextrose with 50 mM Tris, pH 7.4, and 0.9 mM manganese chloride to mimic the reaction conditions of DNase I treatment. One hundred microliters of each sample were extracted once with phenol:chloroform (1:1), extracted twice with chloroform, and 50 μl were loaded on 1% agarose gels in 1X TAE for a load of 1 μg DNA per well.

b. RESULTS

i. Proteoglycans can mediate transfection by CLDC in vitro

Raji cells stably expressing syndecan-1 were transfected by CLDC in a manner dependent on the net positive charge of the complex. This indicated that the syndecan-1 proteoglycan functions in the uptake of CLDC in vitro. Wild type Raji cells were essentially untransfectable by CLDC. However, wild type Raji cells that had been stably transfected with the syndecan-1 gene were much more efficiently transfected by CLDC. Furthermore, CLDC-mediated transfection of syndecan-1 -bearing cells increased significantly as the complex became more positive in net charge. Cells were transfected with CLDC made at ratios of 1:1, 1:2, 1:4, and 1:6 μg DNA/nmole total lipid, and the greatest difference in CLDC-transfection efficiency between wild type and syndecan-1 stable transfectants was seen with CLDC made at a ratio of 1:6. These data showed that, in Raji cell culture, proteoglycan expression is crucial for efficient CLDC-mediated transfection.

ii. Fucoidan inhibits in vitro CLDC-mediated transfection

CLDC uptake is likely to be mediated by the interaction of the positive cationic head group of CLDC with a negatively charged cell surface molecule. To test this mechanism, cells were pretreated with polyanionic compounds prior to transfection, which should interfere with transfection. Pretreatment of cells with either fucoidan, heparin, or dextran sulfate, all polysulfated polysaccharides, largely blocked CLDC transfection in vitro. Both heparin and heparin sulfate have previously been reported to block transfection mediated by poly-lysine:DNA complexes (Mislick and Baldeschweiler, 1996). Concentrations of fucoidan, heparin, or dextran sulfate as low as 100 nM were sufficient to block transfection of murine B 16 cells. Dextran sulfate, which blocked transfection at 10 nM, was the most efficient inhibitor of CLDC transfection. The charge of the polysaccharide appeared to be the significant factor in inhibiting CLDC transfection. Uncharged dextran, with a molecular weight similar to that of the fucoidan, failed to inhibit transfection, and even caused a significant elevation of transfection at high concentrations. Similar levels of inhibition of CLDC-mediated transfection by fucoidan were observed in PPC-1, MDA-435, DU-145, and CHO cells indicating that this is a generalized effect. A concentration of fucoidan (10 nM) that has little effect on CLDC-mediated transfection was, nevertheless, more effective at inhibiting transfection when CLDCs were pretreated instead of cells indicating that these anionic compounds block transfection by binding to cationic head groups on CLDC. Furthermore, fucoidan inhibited the transfection of cells only when using methods relying on positive charge, i.e. CLDC and calcium phosphate. Fucoidan did not interfere with adenoviral or electroporation methods of transfection in vitro, indicating that these approaches function by different pathways of entry.

Fucoidan inhibited CLDC-mediated transfection in vitro by blocking DNA uptake by cells. Nuclei were isolated from cells untreated or pretreated with fucoidan and subsequently transfected with CLDC, and nuclear DNA was assayed for the presence of the luciferase plasmid used in making the complex. The nuclei of cells untreated with fucoidan contained luciferase plasmid, as expected from the high levels of luciferase activity observed in untreated cells. Therefore, DNA did traverse the cell membranes of untreated cells. In comparison, cells pretreated for 30 minutes with 1 μM fucoidan did not express luciferase and had no luciferase DNA in their nuclei. Southern analysis of total DNA from whole cell extracts failed to show evidence of any DNA crossing the cell membranes of cultured cells pretreated with fucoidan. Pretreatment of cells with sodium chlorate, which inhibits glycosaminoglycan sulfation, has previously been shown to decrease DNA binding to cell membranes (Mislick and Baldeschweiler, 1996).

DOPE or cholesterol containing cationic liposomes were capable of protecting DNA from digestion, even in the presence of fucoidan, making it unlikely that disruption of the CLDC is the mechanism by which fucoidan inhibits both DNA uptake and subsequent reporter gene expression in cells. Previously, dextran sulfate has been shown to disrupt DNA complexed to liposomes composed purely of cationic lipid. In order to determine whether DNA was also released from CLDC prepared from a 1:1 cationic lipid:DOPE mixture, CLDC were incubated for 10 minutes with 1 μM fucoidan and subjected to nuclease digestion for 5, 30 and 90 minutes. No detectable degradation of DNA was observed in CLDC pretreated with fucoidan. Conversely, unprotected DNA was readily degraded by a 90 minute exposure to DNase I. Similar liposome-mediated protection of DNA was observed when CLDC were preincubated with dextran sulfate. Multilamellar vesicles of DOTIM:cholesterol were equally capable of protecting DNA in complexes from DNaseI digestion (data not shown). However, CLDC made with pure cationic lipid (DOTAP alone) did not protect DNA from DNaseI digestion with or without fucoidan pretreatment.

(1) Fucoidan inhibits CLDC-mediated transfection following intravenous injection into mice

Fucoidan appeared to inhibit CLDC mediated transfection by the same mechanism in vivo as in vitro. Mice were pretreated with fucoidan for various time periods before intravenous injection of CLDC. Twenty-four hours after CLDC injection, tissues were assayed for luciferase activity and compared to the levels of luciferase expression in tissues from non-fucoidan treated mice. Lung and heart tissues, the tissues most efficiently transfected by intravenous injection of CLDC containing DOTIM:Chol MLV, showed drastic reductions in luciferase activity after mice were pretreated for 1 hour with fucoidan. Longer pretreatments with fucoidan resulted in less significant effects on CLDC-mediated transfection efficiency, presumably due to ongoing clearance of the highly negatively charged fucoidan from the circulation. The magnitude of luciferase depression in tissues pretreated for 1 hour with fucoidan in vivo is similar to that seen in vitro, i. e., approximately 100 fold. Southern analysis of total DNA from mouse lungs and livers showed decreased levels of p4241 plasmid in the tissues of animals pretreated with fucoidan for 1 hour. Nuclear DNA isolated from lungs also showed a 3-fold decrease in p4241 plasmid present in fucoidan treated animals compared to untreated animals. One hour pretreatment with fucoidan also resulted in significantly decreased levels of rhodamine-lipid found in the lung and liver, as assayed by extraction of total lipids from tissues and fluorometric quantitation of the Rh-PE-MLV. These data are consistent with in vitro observations and suggest that fucoidan pretreatment resulted in lower levels of DNA delivered into cells by CLDC which lead to significantly reduced luciferase expression in fucoidan treated animals in vivo.

(2) Proteoglycans are involved in CLDC transfection in vivo

Pretreatment of mice with heparinase I prior to intravenous CLDC injection resulted in significantly lowered levels of luciferase expression, indicating that proteoglycans are important for intravenous CLDC transfection. Heparinase I specifically cleaves the heparin sulfate glycosaminoglycan chains on cell surface proteoglycans, and intravenous injection of heparinase I has been shown to significantly reduce proteoglycan levels in mice. Mice were pretreated with heparinase I by intravenous injection of a saline solution of the enzyme 15 minutes before CLDC injection. Control mice were pretreated for 15 minutes with either saline solution alone or with boiled heparinase I. The transfection efficiency of CLDC was significantly decreased in the lungs, hearts and spleens of heparinase-I-treated mice when compared to the transfection efficiency in tissues from untreated mice. Boiling and denaturing the heparinase I negated the effect of active enzyme on CLDC transfection efficiency in mice, indicating that heparin sulfate cleavage function was necessary to inhibit CLDC-mediated transfection. Pretreatment of mice with a mixture of heparinase I and heparinase III showed the same inhibition of luciferase expression by CLDC transfection in comparison to mice pretreated only with heparinase I. Similar to the effect of fucoidan, heparinase I pretreatment of mice also significantly decreased the levels of rhodamine labelled lipid recovered from lungs in pretreated mice compared to untreated mice. Southern analysis showed 2-fold less reporter plasmid DNA in lungs from mice pretreated with heparinase I when compared to DNA levels found in control lungs. These results indicate that mice pretreated with heparinase I were compromised in their ability to take up DNA delivered by CLDC, and intact heparin and heparin sulfate glycosaminoglycans on the cell surface play a significant role in CLDC-mediated intravenous transfection in vivo.

C. DISCUSSION

Factors which appear to function by a common pathway in mediating CLDC-based gene delivery both in vitro and in vivo are especially important to identify in order to understand, control and improve CLDC-based gene delivery. Recent data in our laboratory highlight the inability to predict consistently and accurately from in vitro results the factors involved in controlling in vivo CLDC-mediated gene transfer. Specifically, depletion of sialic acids, the other predominant anionic cell surface molecule, blocks in vivo transfection by CLDCk, just as depletion of proteoglycans does. Conversely, depletion of proteoglycans in vitro blocks CLDC-based transfection; whereas depletion of sialic acids enhances it in vitro. This lack of correlation between in vitro and in vivo effects demonstrates the importance of confirming in vitro results in in vivo systems. We have identified proteoglycans as an important mediator of both in vitro and in vivo CLDC-mediated transfection.

The role of proteoglycans in mediating the delivery of DNA by cationic liposomes in vivo, and whether that role is inhibitory or supportive, has been a subject of controversy. Experiments using both poly-lysine:DNA and cationic liposome:DNA complexes indicate that proteoglycans assist in the delivery of genes in vitro (Mislick and Baldeschweiler, 1996). Transfections of CHO mutant cells deficient in the display of proteoglycans on the cell surface and cells treated with sodium chlorate to replace the sulfate moieties on the cell surface were less efficient, indicating that proteoglycans and the sulfates on glycosaminoglycans function in the delivery and expression of DNA in vitro (Mislick and Baldeschweiler, 1996). In contrast, results have also been presented showing that polyanionic polysaccharides, including dextran sulfate and heparin, are capable of disrupting CLDC in vitro. It was hypothesized that charge interactions disrupt the structure of CLDC and result in the release of DNA from the complex. Based on these findings, Szoka and colleagues (1996) predicted that proteoglycans on the cell surface might hinder the uptake of DNA in vivo, because proteoglycans display polysulfated glycosaminoglycan chains similar to the polyanionic polysaccharides heparin and dextran sulfate. Furthermore, results of in vitro CLDC-based transfection studies have proven notoriously poor predictors of in vivo results.

In contrast to previous results obtained using liposomes composed of 100% cationic lipid to make CLDC (Xu and Szoka, 1996), the present results indicate that polysulfated polysaccharides do not disrupt complexes made of equal molar amounts of cationic and neutral lipid in vitro.

In addition, the studies using heparinase I and III, enzymes specific for the cleavage of heparin and heparin sulfate proteoglycans, showed that intact proteoglycans are necessary for the efficient delivery of DNA to cells in the tissues of mice injected intravenously with CLDC. Additionally, Raji cells, which are poorly transfected by CLDC, require expression of syndecan-1 to make them transfectable by CLDC-mediated gene transfer.

Consistent with the role of proteoglycans in CLDC-mediated transfection, in vivo fucoidan pretreatment inhibits the expression of luciferase in mice intravenously injected with CLDC. Similarly, heparin abolished CLDC-mediated gene transfer following intravenous injection of CLDC into mice as effectively as fucoidan. Both fucoidan and heparin could bind to positively-charged CLDC, inhibiting binding to negatively charged cell surface proteoglycans. In agreement with this hypothesis, the pre-incubation of CLDC with fucoidan blocked transfection in vitro more efficiently than pre-incubating cells with fucoidan. Alternatively, the large polyanionic polysaccharides fucoidan and heparin, resembling extracellular matrix components, could complex with cellular proteoglycans rendering the proteins unavailable for binding to CLDC.

The inhibitory effect of the specific enzymes, heparinase-I and-III, on gene expression in tissues of mice following intravenous injection of CLDC indicates the specific involvement of proteoglycans, or at least the glycosaminoglycan chains of these cell surface proteins, in the uptake of CLDC into cells. Cleavage and release of glycosaminoglycan chains yield at least two possible mechanisms for the inhibition of CLDC uptake into cells by heparinase pretreatment. First, cells stripped of glycosaminoglycan chains by pretreatment with heparinase would be devoid of negatively charged ‘CLDC-receptors’ and consequently would be unable to bind CLDC. Alternatively, the glycosaminoglycan chains released by enzymatic cleavage in the tissues of animals pretreated with heparinase could possibly bind to CLDC and prevent the complex from contacting the appropriate‘receptor’ on the cell surface. In either scenario, CLDC must bind to polyanionic glycosaminoglycan chains, suggesting that the glycosaminoglycan portion of the proteoglycan is the initial binding site for CLDC in vivo.

After binding, the precise role of proteoglycans in mediating CLDC uptake into cells both in vitro and in vivo remains to be elucidated. Proteoglycans could bind CLDC and then be internalized as a proteoglycan:CLDC complex into cells. Alternatively, proteoglycans could initially bind CLDC and present the complex to a second cell surface protein or receptor, which in turn undergoes endocytosis, similar to the involvement of proteoglycans in mediating the internalization of lipase. There is at least one example of a proteoglycan requiring other proteins to undergo endocytosis: two receptors of 51 and 26 kD mediate the binding and endocytosis of decorin, a plasma proteoglycan (Gotte et al., 1995). In view of potential involvement of unidentified proteins in CLDC uptake, it is interesting to note that fucoidan is an inhibitor of the scavenger receptor. This suggests the possibility that the scavenger receptor can play a role in CLDC uptake.

One can conclude from this and other studies that the proteoglycan superfamily serves as the major receptor for all gene delivery vectors that produce cationic DNA complexes. This being so, the interaction of the complex with the cell is primarily electrostatic, and does not involve a receptor specificity of the binding site for the cationic moiety. Therefore, the substantial differences in transfection efficiency between various cationic systems are most likely caused by differences in physical properties of the complex such as size, stability, net surface charge, or charge density. This inference is important because it points to the most fruitful area for the future development of these systems.

Identification of proteoglycans as the CLDC receptor in vivo represents an important breakthrough in the study CLDC-mediated gene transfer. For example, the introduction heterologous genes can allow for the manipulation of proteoglycan expression or function in vivo in order to control the process of CLDC-mediated gene transfer in vivo. Given the presently described in vivo results, one can effectively modulate CLDC-mediated gene transfer in vivo. For instance, one can up modulate CLDC transfection in vivo by singly or multiply pretreating the host animal with polynucleotides encoding proteoglycan receptors in order to increase the amount of proteoglycans on the surface of host animal cells. Such proteoglycan enhanced cells will be more effectively transfected by the presently described methods and vectors. Conversely, one can down modulate CLDC mediated gene transfer by treating the host animal with heparinase (to remove proteoglycan receptors) or fucoidan (which competitively inhibits proteoglycan binding to CLDC). Moreover, as host cells and tissues vary in their rate of recovery from heparin or fucoidan treatment, such treatments also allow for effective targeting of specific animal cells, organs, or tissues. For example, at 10 hours post exposure, fucoidan still inhibits CLDC mediated transfection of lung cells, whereas liver cells can be durably transfected using CLDC-mediated gene delivery.

### Example

### Anti-Cancer Gene Therapy

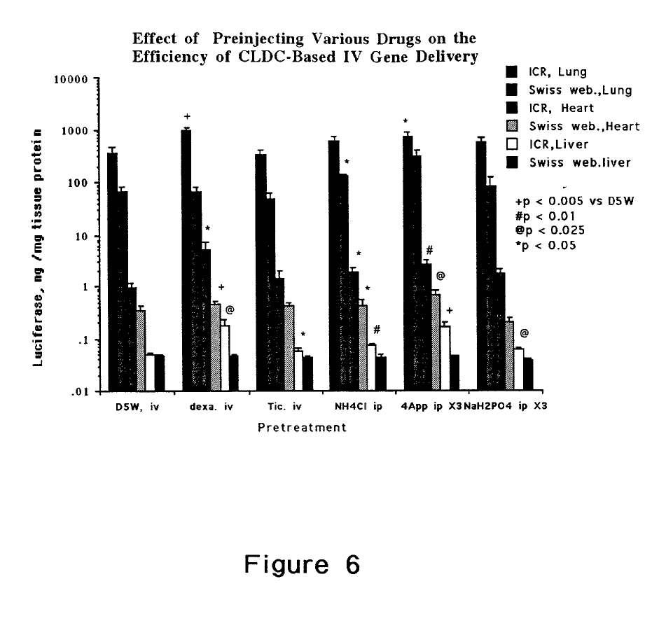

B16 Melanoma-induced tumors were used to assess the potential of CLDC mediated cancer therapies. C57 Black 6 mice were i.v. (tail vein) injected with 25,000 syngeneic B16-F-10 melanoma cells. CLDC were prepared essentially as described above using 25 μg of a HCMV-driven expression plasmid (p4109, Liu et al., 1995, J. Biol. Chem., 270(42):24864-24860) into which either the murine angiostatin gene, the murine GM-CSF gene, the human p53 gene, or the CAT gene (for use as a mock treated control) had been subcloned. CLDC were i.v. injected three days after initial tumor challenge, and again on day 10. The mice were sacrificed on day 30 and the total number of lung metastases, and number of metastases>2 mm were counted using a dissecting microscope. These data are presented in FIG. 7. Relative to non-treated control mice or the CAT-CLDC treated control mice, the test mice that had been treated with CLDC containing the murine angiostatin gene, the murine GM-CSF gene, or the human p53 gene (the “test CLDC”) produced significant anti-metastatic effects (greater than 50% reduction in observed metastases, p<0.05). Similar, results were obtained when the total number of blood vessels/tumor were quantified (vessels were stained using a FVIII, anti-VWF antibody). When different combinations of test CLDC were used to treat animals, further, but only marginally significant, reductions were observed in mixed compositions comprising the GM-CSF gene. The tumor studies clearly indicate that even early generation CLDC/vectors can deliver and express therapeutically relevant concentrations of desired biological products. These data demonstrate that the presently described methods and tools can be implemented to provide significant anti-metastatic tumor activity via the systemic delivery of anti-angiogenic genes such as angiostatin, endostatin, GM-CSF, or p53.

Additional studies using groups of 5, C57BL6 female mice that received a single tail vein injection of CLDC containing either 25 μg of the CMV-P53 expression plasmid, 25 μg of a CMV-luciferase expression plasmid (mock-treated), or no treatment (control). All mice were sacrificed 1 day after i.v. injection of CLDC, and their lungs were then removed and microscopic sections analyzed for expression of the human p53 gene by standard immunohistochemical procedures by an investigator who was unaware from which treatment groups the mice came. The presence of p53 antigen is indicated by the reddish staining cells, and melanin-containing tumor cells stain dark brown.

Results: Lungs from mice injected with CLDC containing the CMV-p53 expression plasmid show positive staining for the p53 antigen in approximately 20% overall of B-16 melanoma cells metastatic to lung as well as significant numbers of normal lung cells. Further observations showed widespread p53 antigen positivity in the tumor cells from p53 gene-treated mice. Neither lungs from the CMV-luciferase expression plasmid treated mice (mock-treated controls) or untreated mice exhibited significant positive staining for p53 gene expression. Thus, i.v. injection of CLDC containing the human wildtype p53 gene transfects large numbers of metastatic tumor cells with p53.

### Example

### Identification of Novel In Vivo Gene Function for GM-CSF

By employing simultaneous gene co-delivery to establish novel functionality, it was discovered that the GM-CSF gene can mediate significant antiangiogenic anti-tumor activity by codelivering the GM-CSF gene with a gene known to produce antiangiogenic anti-tumor activity in tumor-bearing animals (O'Reilly et al., 1997). Specifically, we tested whether co-injection of the angiostatin and GM-CSF genes into individual groups of mice produced additive or synergistic anti-tumor activity when compared to injection of the individual genes alone. CLDC-based i.v. injection of each gene individually, as well as of the genes in combination reduced both the total number of lung tumors and the numbers of tumors greater than 2 mm by comparable levels when compared to control mice (data not shown). Thus, the combination of genes did not enhance the level of anti-tumor activity when compared to that produced by each gene individually, indicating a clear lack of additive or synergistic anti-tumor activity. These results indicated that each gene is acting via a common anti-tumor pathway.

The antiangiogenic function of GM-CSF was confirmed by performing intratumoral blood vessel counts (see below) to quantitatively compare how the expression of angiostatin and/or GM-CSF effected tumor blood vessel formation in vivo. These studies confirmed that CLDC-based delivery of the angiostatin and GM-CSF genes each produced highly significant and comparable reductions in tumor neovascularity when compared to control mice (p<0.005) (see Table 3), documenting that each gene product induced highly significant and similar anti-angiogenic activity within tumors in tumor-bearing mice.

TABLE 3
Intravenous, CLDC-based injection of the angiostatin or GM-CSF genes
each significantly reduces tumor vascularity.

| | Total # blood |
|---|---|
| CLDC injected | vessels/tumor |
| CLDC-angiostatin gene | 7.7 ± 2.1* |
| CLDC-GM-CSF gene | 8.5 ± 3.4⁺ |
| CLDC-CAT (Control) | ’14 ± 3 |
| CLDC-p53 | 7.9 ± 2.3* |

Mice were treated as described in FIG. 1b.
Vessels were stained using a FVIII, anti-VWF antibody
*p <0.0005 vs control
⁺p <0.01 vs control

### Example

### Identification of Novel In Vivo Gene Function for CC3

The present invention can also be employed to identify new and unanticipated gene functions—functions unrelated to the specific functions previously assigned/identified for a given gene. For example, the CC3 gene has been identified as a metastasis suppressor gene whose loss of function produces an aggressive metastatic phenotype. This occurs only in cells that have lost both copies of the wildtype gene, and therefore produce no wildtype protein (E. Shtivelman, 1997, Oncogene, 14:2167-2173). Loss of function of the CC3 gene leads to an aggressive metastatic phenotype that occurs in a subset of highly metastatic cancers of neuroectodermal origin. CC3 should not produce anti-tumor effects against melanoma tumors because wildtype CC3 is present in human melanomas and because suppressor genes only give rise to tumors when the function of the wildtype gene product is lost. Unexpectedly, CLDC-based i.v. gene delivery of the wildtype human CC3 cDNA produced significant anti-metastatic tumor effects against B16 melanoma in tumor-bearing mice (FIG. 8). This result was unexpected because B16 melanoma cells already express the endogenous wildtype CC3 gene product, and the anti-tumor function previously identified for CC3 is as a tumor suppressor gene (E. Shtivelman Oncogene, 1997). By definition, since the endogenous wildtype CC3 gene product is present in the B-16 tumors in mice bearing these tumors, CC3 is not functioning as a specific tumor-suppressor gene.

### Example

### Single Plasmid In Vivo Expression

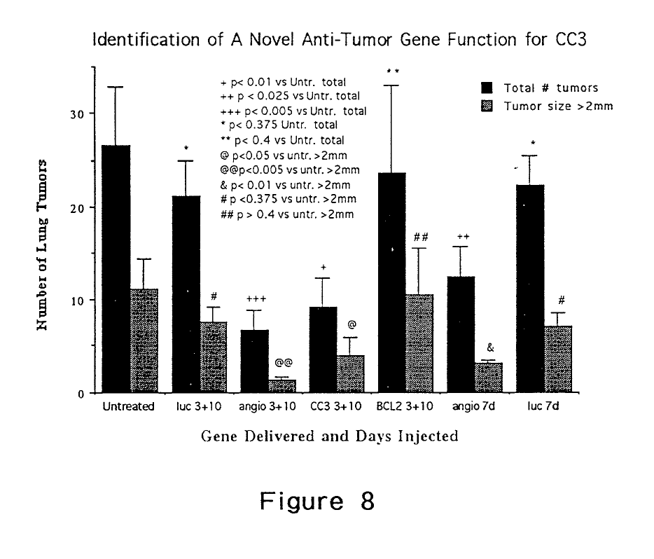

A single expression plasmid containing multiple, independent and functional expression cassettes can also be used to produce long-term, high level expression of multiple different genes following CLDC-based in vivo delivery of the single plasmid. Specifically, expression plasmid p4458 contains both a complete HCMV-luciferase cDNA plus EBV family of repeats (FR) expression cassette and a complete HCMV-EBNA-1 cDNA expression cassette. p4458 produces long term, high level luciferase gene expression following CLDC-based iv injection into animals. The plasmids used for this experiment are diagrammed on FIG. 9; their construction is described below.

To produce p4458, the following constructs were made.

An approximately 2 kb fragment (bp 839-2873) containing the EBNA-1 cDNA from p630 (Middleton and Sugden, 1994, supra.) was excised with Hind III and Acc I and ligated into the Hind III-Acc I site of vector p4109 (Liu et al., 1995, supra.) to form plasmid p4331. In addition, vector pVR1255 (Hartikka et al., 1996, Hum. Gene Ther. 7:1205-1217) was digested with Eco RV and Bam HI, end-filled, and then the same 2 kb EBNA-1CDNA, excised from p630, was end-filled and ligated into pVR1255 to form plasmid p4395 (CMV-EBNA-1).

Plasmid p4329 was constructed by partially digesting p985 (Middleton and Sugden, 1992, supra.) with Bam HI, followed by Kpn I, and ligating the approximately 3 kb family of repeats (FR)+TK promoter+Luciferase containing fragment (bp 1099-4043) into the Bam HI and Kpn I sites of plasmid p4109 (Liu et al., 1995).

Plasmid p4379 (CMF-luc-FR-2) contains the approximately 900 bp family of repeats fragment (bp 3157-4043), isolated from p985 by Bam HI digestion followed by insertion into the Bam HI site of vector pVR1255. Thus, the FR is located downstream from the luciferase gene.

p4458 is based on p4379, which was digested with Xmn I and end-filled. The 3.5 kb fragment containing the full p4379 expression cassette (CMV-intro-EBNA-1-poly A fragment) was excised from p4331 with Xho I+Bgl II, end-filled, and subsequently ligated into the Xmn I site of p4379 to form p4458, a single plasmid containing CMV-CMV-EBNA-1 CMV-luc-FR-2.

Plasmids were purified using alkaline lysis and ammonium acetate precipitation as described (Liu et al., 1995, supra.).

A single CLDC-based intravenous injection of p4458 produces long term expression (>12 weeks) of the luciferase gene in immunocompetent mice. We have previously shown that following co-injection of our EBV-based two plasmid system, both the HCMV-luciferase cDNA plus FR expression plasmid and the HCMV-EBNA-1 cDNA expression plasmid must each be expressed in order to produce long term expression of the luciferase gene. Thus, both expression cassettes, CMV-luc-FR and CMV-EBNA-1, are expressed in vivo by the single plasmid, p4458. The use of a single expression plasmid that contains multiple functioning expression cassettes, including an FR-containing expression cassette and an expression cassette driving the EBNA-1 cDNA creates the ability to express multiple genes at biologically and therapeutically significant levels for the extended periods of time necessary to produce distinct phenotypes following non-viral-based gene delivery in vivo. This approach substantially increases the number of genes and cDNAs that can be delivered and evaluated in order to identify gene function in individual animals. This is particularly important since high DNA doses, which would be necessary to co-deliver multiple different expression plasmids in individual animals often prove too toxic or lethal. Therefore, the use of single, long expressing plasmids containing multiple different cDNAs/genes in multiple expression cassettes will make the assessment of functional genomics following non-viral in vivo gene delivery into animals both scientifically and economically feasible. Furthermore, since CLDC-based gene delivery is capable of delivering megabase size pieces of DNA into cells (J. Harrington et al., 1997, Nat Genet. 4:345-355), the presently described approach can be used to deliver very large (≧30 kb) sized-plasmids into mice. In vivo delivery and expression of large plasmids had not been accomplished to date. Here, studies are described that demonstrate that a 32.1 kb expression plasmid containing the luciferase cDNA can be intravenously injected and efficiently expressed in mice.

An additional embodiment of the present invention includes a single, or multiple, plasmid expression system that transiently expresses the CRA (for example, EBNA-1) initially, but incorporates control elements that allow for inhibiting the expression of the CRA in a regulatable fashion. Examples of such mechanisms include, inducible promoters that will not appreciably express a given gene absent the proper inductive signal (e.g., but not limited to, heavy metal inducible promoters, steroid dependent promoters/control elements, transcription factor dependent promoters, etc.), or by engineering CRA expression such that it can be shut down by specific stimuli such as, but not limited to, regulatable antisense inhibition of expression, or the incorporation of recombinase control elements flanking the CRA coding region that allow for the “flipping” or deletion of CRA in the construct in order to shut-off CRA expression.

### Example

### Functional Genomics

The present invention enables a functional assessment of any non-viral/CLDC-delivered gene of unknown function. As demonstrated by the GM-CSF data, the present invention can also be used to identify new and unanticipated functions of genes in addition to those previously identified either biochemically or phenotypically.

A typical functional genomic assay is conducted in the following manner:

Following a single in vivo (i.v., aerosol, intra-CNS, IP, in utero or other) injection of CLDC or other non-viral approach, unknown gene- and/or cDNA-injected animals are compared to mock-treated controls (vector containing a non-coding sequence or reporter gene) and untreated control groups and monitored closely for any behavioral or activity changes, abnormal gain or loss of weight, eating and drinking changes. Additionally, the progression, amelioration, or prevention of disease states can be monitored using suitable genetically modified germline transgenic animal models, naturally occurring genetic models or normal mice. Using this approach, a variety of parameters are monitored, for instance, in the transgenic animal models, including animal models of cancer, heart disease, atherosclerosis, hypertension, diabetes, asthma, obesity, neurodegenerative diseases, etc. Among the parameters closely screened in multiple are clinical appearance (skin, hair, bodyweight etc.), full bloodcounts and blood chemistries, hormone levels, cytokine levels, induction of specific or non-specific immune responses, induction of immunotolerance, full histopathologic analysis, including monitoring for possible target organ injuries and inflammatory responses. The assessment of parameters such as these in animal disease models will maximize the amount of functional information derived per treated animal, and will include direct functional information on whether the unknown genes produce significant therapeutic effects in animal models directly relevant to common diseases.

In addition, the present invention provides functional information on whether the unknown genes function as growth factors, colony stimulating factors, cytokines, chemokine agonists or antagonists, etc. Further, these same studies can be performed in normal mice to determine whether the unknown gene functions to induce specific disease states, including cancer, heart disease, atherosclerosis, hypertension, diabetes, asthma, obesity, neurodegenerative diseases, etc., as well as whether they function as growth factors, CSFs, cytokine, chemokine agonists or antagonists etc. in normal hosts. By correlating the observed phenotypic changes with the introduction of specific cDNAs (by comparing the treated animals with both mock-treated and untreated control animals) the specific function(s) of these uncharacterized DNA sequences can be assessed. This approach is of greatest utility for full length cDNAs or genomic clones, or for partial clones from which full length clones can be generated.

The present invention can also be used to target phenotypic markers based on an anticipated gene function. For example, evolutionary genes capable of erythropoietic activity can be targeted by focused screening for phenotypes related to anticipated or desired endpoints such as the elevation of hematocrit, lymphocyte counts, or targeted enzymes such MnSOD, etc. This focused screening enhances throughput where gene function can be hypothesized or more closely categorized.

### Example

### Tandem Enhancer/Promoter Elements

In this example, the effect of tandem promoters directing expression of luciferase was examined in vivo. Expression plasmids were administered in CLDCs to mice as described above in Sections 6 and 14.

a. Vector construction

p4531 (hm CMVSA11) was constructed by isolating the 1.3 kb XbaI-EcoRI fragment of pMH5 (purchased from Microbix Biosystems, Inc.) CMV enhancer/promoter element and ligating it into the SacII site of pVR1255 (Hartikka et al., 1996. Human Gene Therapy. 7:1205-1217) by blunt end ligation. p4610 (hm 4CMVSA23) containing a composite hCMV and short mCMV enhancer/promoter was constructed by ligating the 529 bp Bst1 1071-EcoRI fragment from pMH5 into p4377 at the SacII site by blunt-end ligation.

p4588 was constructed by blunt end ligation of the 1 kb Spel-HindIII fragment of the FLT-1 5′ UTR (−748/+284) linked to luciferase DNA (Morishita et al., 1995. J. Biol Chem. 270:27948-27953) into pVR1255 at the PstI site. The construction of p4590 was similar to that of p4588, but the 1 kb Spel-HindIII fragment was ligated into hmCMVSA11 at PstI. Plasmid p4377 (also known as pVR1255), was used as a control and contains an optimized hCMV enhancer/promoter, intron A, kanamycin resistance gene, and polyA termination signal.

b. Results

Expression of luciferase from plasmid pmhCMVSA11 in lung, heart and spleen was dramatically increased (from 6 to almost 20 fold) over that from the luciferase expression plasmids p4377 (containing a single copy of the optimized human CMV promoter) and pmCMVSA2 (containing a single copy of the mouse CMV promoter). Thus, the tandem enhancer/promoter elements functioned synergistically to increase levels of gene expression.

The Flt-1 promoter is a tissue specific promoter expressed specifically in vascular endothelial cells. Addition of mCMV enhancer/promoter and/or the hCMV enhancer/promoter increased tissue specific expression of luciferase when operatively linked to the Flt-1 promoter driving a luciferase expression plasmid. These results indicate that multimers of enhancer/promoters can be used to increase expression of a desired gene of interest in vivo while retaining cell type and tissue specificity.

### Example

### Formulation of Naked Polynucleotide Complexes

In general, transgenic delivery (and subsequent expression) of naked DNA is less efficient that of optimized CLDC. Accordingly, formulations of naked DNA with various organic molecules was investigated.

Materials and Methods: Expression plasmid pVR1255 was administered to mice at 100 ug in 200 microliters of D5W containing a purified high density lipoproteins (HDLs) fraction isolated according to Fielding et al., Fielding et al., 1986, J. Biol. Chem. 261:5233-5236. Briefly, blood was collected into ice-cooled tubes from normolipemic donors who had fasted overnight. These contained streptokinase (final conc. of 150 IU/ml) as anticoagulant. LDL was affinity-purified from plasma as previously described. All steps were carried out at 2 to 4° C. Briefly, native plasma was passed through columns of heparin-agarose (Sepharose 6B-heparin) (Sigma) equilibrated in Dulbecco's phosphate-buffered saline, pH 7.4 (PBS). In the nonadsorbed fraction, >98% of FC was recovered within density limits 1.063-1.21 gml1. the adsorbed fraction containing LDL was eluted with 3M NaCl/PBS. In the adsorbed fraction, >95% of FC was recovered within density limits 1.019-1.063 gml1. After dialysis overnight at 2-4° C. and concentration into PBS containing 0.05 mM EDTA by centrifugal dialysis, LDL or the nonadsorbed fraction containing HDL was used immediately in individual experiments or frozen or kept at 4° C. All other methods were as described above.

Results: Formulation of naked DNA with a partially purified fraction of high density lipoproteins significantly increased subsequent expression of luciferase. Levels of expression correlated with the concentration of HDL and were increased as much as 40 fold at the highest HDL concentration.

### Equivalents

The foregoing specification is considered to be sufficient to enable one skilled in the art to broadly practice the invention. Indeed, various modifications of the above-described makes for carrying out the invention which are obvious to those skilled in the field of microbiology, biochemistry, organic chemistry, medicine or related fields are intended to be within the scope of the following claims. All patents, patent applications, and publications cited are herein incorporated by reference.

What is claimed is:

1. A method of introducing a gene of interest in an animal cell in vivo, comprising:
a) introducing into an animal a first recombinant polynucleotide sequence encoding a cellular retention activity;
b) introducing into the animal a second recombinant polynucleotide sequence encoding the gene of interest and having a cellular retention sequence,
wherein the cellular retention activity is expressed in the cell and binds the cellular retention sequence, thereby maintaining the second recombinant polynucleotide sequence in the animal cell at least 50% longer than in the absence of the cellular retention sequence.

2. The method of claim 1 wherein the gene of interest is operatively linked to a sequence that directs transcription of the gene of interest.

3. The method of claim 1 wherein the first recombinant polynucleotide sequence encoding the cellular retention activity is introduced on a separate vector from the second recombinant polynucleotide sequence.

4. The method of claim 3 wherein the first and second recombinant polynucleotide sequences are introduced into the animal substantially simultaneously.

5. The method of claim 3 wherein the first recombinant polynucleotide sequence is introduced into the animal 45 minutes to 24 hours before introducing the second recombinant polynucleotide sequence.

6. The method of claim 1 wherein the first recombinant polynucleotide sequence encoding a cellular retention activity and the second recombinant polynucleotide sequence are on the same vector.

7. The method of claim 1 wherein the first and second recombinant polynucleotide sequences are introduced into the animal via a cationic lipid or cationic polymer complex.

8. The method of claim 1 wherein the first and second recombinant polynucleotide sequences are introduced into the animal as naked DNA.

9. The method of claim 1 wherein the animal is a mammal.

10. The method of claim 9 wherein the mammal is a mouse.

11. The method of claim 9 wherein the mammal is a human.

12. The method of claim 1 wherein the cellular retention activity is selected from the group consisting of: EBNA-1, karyopherin, HCMV IE-1, and adenovirus preterminal protein.

13. The method of claim 12 wherein the cellular retention activity is EBNA-1.

14. The method of claim 2 wherein the gene of interest is operatively linked to at least two tandem enhancer/promoter sequences.

15. The method of claim 1 wherein the gene of interest is selected from the group consisting of: angiostatin, endostatin, p53, GM-CSF, IL-2, G-CSF, BRCA1, BRCA2, RAD51, endostatin, TIMP 1, TIMP-2, Bcl-2, and BAX.

16. The method of claim 1, wherein the method is used to determine the in vivo function of a product encoded by the gene of interest.

17. A method of performing functional genomics, the method comprising:
a) introducing into a test animal a recombinant polynucleotide that directs the expression of at least one gene of interest;
b) comparing the phenotype of the animal to a control animal; and
c) identifying differences in the phenotype of the test animal and the control animal.

18. The method of claim 17, wherein the phenotypes of the test animal and the control animal are compared using gene expression profiling.

19. A method of performing functional genomics, the method comprising:
a) introducing into an animal a first recombinant polynucleotide encoding a cellular retention activity;
b) introducing into the animal a second recombinant polynucleotide encoding the gene of interest and having a cellular retention sequence, wherein the cellular retention activity is capable of maintaining the second recombinant polynucleotide having the cellular retention sequence in a cell; and
c) comparing the phenotype of the animal to a control animal.

20. The method of claim 19 wherein the first recombinant polynucleotide encoding a cellular retention activity is introduced on a separate vector from the second recombinant polynucleotide.

21. The method of claim 19 wherein the first recombinant polynucleotide encoding a cellular retention activity and the second recombinant polynucleotide are on the same vector.

22. The method of claim 19 wherein the comparing step comprises gene expression profiling.

23. The method of claim 19 wherein the animal is a mammal.

24. The method of claim 23 wherein the mammal is a mouse.

25. A method of increasing the levels of expression of a gene of interest that is administered to an animal via gene therapy, the method comprising treating the animal with an agent during the 45 minutes to about 72 hours preceding the administration of the gene of interest, wherein the agent increases the subsequent expression of the gene of interest, and administering the gene of interest to the animal via gene therapy.

26. The method of claim 25 wherein the agent is dexamethasone or 4-APP.

27. The method of claim 25 wherein the agent is mannitol.

28. The method of claim 25 wherein the agent induces expression of endogenous proteoglycans or is a polynucleotide encoding a proteoglycan receptor.

29. A method of identifying genetic host factors affecting efficiency of gene delivery, the method comprising:
comparing the expression profile of an animal that has been treated with an agent that increases the levels of expression of a gene of interest in a gene therapy vector with the expression profile of an animal that has not been treated with the agent;
analyzing the difference in expression profiles; and
identifying specific endogenous genes that affect efficiency of gene delivery and/or gene expression.

30. The method of claim 29 wherein the agent is dexamethasone.

31. The method of claim 29 wherein the agent is 4-APP.

32. A method of identifying genetic host factors that affect the efficiency of nonviral gene delivery, comprising:
a) nonvirally delivering a polynucleotide containing a gene of interest to an animal of a first strain in vivo;
b) assessing the levels and extent of gene expression in the first animal;
c) nonvirally delivering a gene of interest to an animal of a second strain in vivo;
d) assessing the levels and extent of gene expression in the second animal; and
e) comparing the genotype of the first strain with the genotype of the second strain.

33. The method of claim 32, wherein the animal is a mammal.

34. The method of claim 33, wherein the mammal is a mouse.

35. The method of claim 34, wherein the first strain is an ICB mouse and the second strain is a Swiss Webster mouse or a FVB mouse.

36. The method of claim 33, wherein the mammal is a human.

37. A method of optimizing delivery and expression of a gene using gene therapy in an animal, the method comprising:
determining the genotype of the animal selected for gene therapy; and
optimizing the delivery method of the gene for the genotype of the animal.

38. The method of claim 37 wherein the animal is a mouse.

39. An episomal vector comprising:
a) a gene of interest;
b) at least two enhancer/promoter regions operatively linked to the gene of interest.

40. The vector of claim 39 further comprising:
c) a cellular retention sequence.

41. The vector of claim 39 wherein one of the enhancer/promoter regions is a tissue specific promoter.

42. The vector of claim 39 that additionally encodes a cellular retention activity that binds to the cellular retention sequence.

43. A cationic molecule/DNA complex, comprising:
a) a biocompatible cationic lipid or a cationic polymer;
b) a neutral lipid; and
c) the vector of claim 39.

44. The complex of claim 43, wherein the cationic lipid cumulatively comprises less than about sixty percent cholesterol or DOPE.

45. The complex of claim 43 that was formed in a solution comprising dextran 40 and Ringers lactate.

46. The complex of claim 43 that was formed in a solution comprising 5 percent dextrose.

47. A polynucleotide composition for gene therapy comprising a purified DNA vector and a purified glycoprotein.

48. The composition of claim 47 wherein the glycoprotein is a high density lipoprotein.

49. The composition of claim 47 wherein the vector contains an expression cassette.

50. A method of inhibiting the growth of a tumor in an animal, the method comprising delivering a polynucleotide that encodes a gene product selected from the group consisting of: angiostatin, endostatin, p53, GM-CSF, TIMP-2, CC3 and BAX to the animal, wherein the gene product is expressed from the polynucleotide and inhibits tumor growth in the animal.
